(19) United States
(12) Patent Application Publication
(10) Pub. No.: US 2024/0130709 A1
(43) Pub. Date: Apr. 25, 2024

(54) SEQUENTIAL ADAPTOR FOR COMBINED ULTRASOUND AND OPTOACOUSTIC DIAGNOSTIC INTERROGATION OF THE LEFT INNOMINATE VEIN

(71) Applicants: The Board of Regents of The University of Texas System, Austin, TX (US); NONINVASIX, INC., GALVESTON, TX (US)

(72) Inventors: DONALD S. PROUGH, III, GALVESTON, TX (US); YURIY PETROV, GALVESTON, TX (US); CHARLES HOUSSIERE, GALVESTON, TX (US); IRENE PETROV, GALVESTON, TX (US); TRIS MILLER, AUSTIN, TX (US)

(21) Appl. No.: 18/538,944

(22) Filed: Dec. 13, 2023

Related U.S. Application Data
(63) Continuation of application No. PCT/US22/33668, filed on Jun. 15, 2022.
(60) Provisional application No. 63/211,541, filed on Jun. 16, 2021.

(51) Int. Cl. A61B 8/00; A61B 5/00; A61B 5/145

(52) U.S. Cl. CPC A61B 8/4236; A61B 5/0095; A61B 5/14542; A61B 8/4416; A61B 8/463; A61B 8/54

(57) ABSTRACT

Apparatus and methods are described for ultrasound guided optoacoustic monitoring to provide diagnostic information for many clinical applications of blood oxygenation in blood vessels and in tissues including for early diagnosis and management of circulatory shock (including that induced by hemorrhage). In certain embodiments provided herein, methods and apparatus for optoacoustics for measurement of blood oxygenation in the innominate vein are provided. In certain embodiments provided herein, are methods and apparatus for articulating an angle between an ultrasound probe and subject body to identify an optimum position for an optoacoustic probe to measure blood oxygenation in a target blood vessel or tissue.

### CROSS-REFERENCE

This application is a continuation of International Application No. PCT/US2022/033668, filed Jun. 15, 2022, which claims the benefit of U.S. Provisional Patent Application No. of U.S. Provisional Application No. 63/211,541, filed Jun. 16, 2021, which is hereby incorporated by reference in its entirety herein.

### STATEMENT AS TO FEDERALLY SPONSORED RESEARCH

This disclosure was made with government support under Contract 1R41HD094542-01A1 (“Noninvasive Monitoring of Total Hemoglobin Concentration in Neonates”) awarded by the National Institute of Health (NIH), an agency of the U.S. Department of Health and Human Services. The government has certain rights in the invention.

### BACKGROUND OF THE INVENTION

Provided herein are apparatus and methods for measurement of blood oxygenation in the major veins. The background is described in connection with existing methods for measurement of blood oxygenation in major veins and in particular the innominate or brachiocephalic vein.

Civilian trauma is similar to military trauma with leading cause of death and morbidity due to hemorrhage with and without traumatic brain injury (TBI). Potentially survivable injuries from hemorrhage require rapid assessment and treatment. Prompt triage from point of injury to definitive care has been shown to improve outcome. However, rapid triage is rarely feasible in combat casualty care. Thus, improvements in prolonged field care (PFC), which is up to 72 hr. combat casualty care in austere environments, are essential. Potentially lethal injuries, e.g. hemorrhage with and without TBI, must be effectively managed during this prolonged and critical period, which necessitates precise resuscitation in order to prevent sequelae of over- and under-resuscitation. The use of vital signs, e.g., blood pressure, etc., to guide resuscitative efforts in hemorrhage have poor predictive value, especially in the young and healthy. TBI in combination with hemorrhage may further confound vital sign interpretation. This is especially problematic since hypovolemia dramatically worsens outcome in TBI victims.

Outcomes may dramatically worsen if TBI is not recognized. Currently, the primary indices used to diagnose and monitor treatment of hemorrhagic shock are blood pressure, heart rate, and mental status, which are relatively nonspecific and insensitive. Further, those indices could be relatively normal despite ongoing tissue hypoperfusion. No rapidly available noninvasive diagnostic test is available to detect systemic hypoperfusion in subjects in whom blood pressure and heart rate are grossly normal.

Although supplemental monitoring may detect tissue hypoperfusion and guide resuscitation efforts, the only measurement shown to improve outcome in circulatory shock is central venous (superior vena cava (SVC)) hemoglobin saturation (ScvO₂). Early goal-directed therapy (EGDT) resuscitation of hypovolemic septic shock, guided by targeting ScvO₂, reduced both mortality (46.5% to 30.5%) and hospitalization cost. See Rivers E, et al. Early Goal-Directed Therapy in the Treatment of Severe Sepsis and Septic Shock. N Engl J Med. 345 (2001) 1368-1377. ScvO₂ has also been proposed as a prognostic indicator in several pathological conditions, including polytrauma subjects. Low ScvO₂ in major trauma and head injury subjects was associated with higher mortality and prolonged hospitalization. Measuring ScvO₂ in the critical interval from point-of-injury to definitive care would improve diagnosis of early shock and enable monitoring of therapeutic interventions. However, central venous catheterization is invasive, time-consuming, complication-prone and challenging in resource-constrained conditions.

The apparatus and methods herein for non-invasive monitoring of ScvO₂ would provide a long-needed solution to support and direct EGDT.

### SUMMARY OF THE INVENTION

Provided herein are methods and apparatus for ultrasound-guided optoacoustic measurement of blood oxygenation in a blood vessel. In certain embodiments a site for monitoring the blood vessel is first identified using an ultrasound probe, and subsequently, an optoacoustic stimulus and detector is utilized at the identified site and blood oxygenation is measured in venous blood carried by the blood vessel using the optoacoustic stimulus and detector. In certain embodiments, the blood vessel is selected from the innominate vein, the internal jugular vein, the subclavian vein, and the femoral vein.

In particular embodiments the blood vessel is the innominate vein. In certain embodiments the site is located using a subject interface through which the ultrasound probe is first removably applied to locate the blood vessel followed by removal of the ultrasound probe and application of the optoacoustic probe. In other embodiments, the ultrasound probe and the optoacoustic stimulus and detector are mounted together in a holder and once the blood vessel of interest is located with the ultrasound probe, optoacoustic stimulation is delivered, and measurements are performed with the optoacoustic detector. In such embodiments the ultrasound locating and optoacoustic measuring may be performed simultaneously and continuously.

In certain embodiments of the method an axis of the optoacoustic stimulus is parallel to an axis of the ultrasound probe while in other embodiments an axis of the optoacoustic stimulus is adjusted at an angle with respect to an axis of the ultrasound probe to provide accurate probing from a specific depth in the blood vessel.

In certain embodiments, the ultrasound locating and optoacoustic measuring are performed using the same ultrasound probe.

In certain embodiments, the optoacoustic stimulus is provided with at least a pair of wavelengths selected from: 760 nm and 800 nm; 1064 nm and 800 nm; and 760 nm and 1064 nm.

In certain embodiments, a subject interface for ultrasound guided optoacoustic measurement of blood oxygenation in a blood vessel is provided including a holder that is dimensioned to securely hold an ultrasound probe, and a subsequently applied optoacoustic probe to a site on a subject where the ultrasound probe is able to detect a major vein and the optoacoustic probe is able to detect blood oxygenation in the detected major vein.

In other embodiments an apparatus for ultrasound guided optoacoustic measurement of blood oxygenation in a blood vessel is provided that includes a housing that is dimensioned to hold an ultrasound probe, an optoacoustic probe, and a light source securely and simultaneously for generating optoacoustic waves at a site on a subject where the ultrasound probe is able to detect a major vein and the optoacoustic probe is able to detect blood oxygenation in the detected major vein. The housing may further include a gel cavity that is adapted to hold an acoustic gel that directly communicates a face of the ultrasound probe and face of the optoacoustic probe to the skin of a subject. In certain embodiments, the housing includes a gel-filled tube that provides for filling and maintaining a fill of the gel cavity. The housing may direct an axis of the ultrasound probe and an axis of the optoacoustic probe in parallel or at an angle to each other. The light source may be an optical parametric oscillator (OPO), laser diode, light emitting diode (LED), pulsed laser diode, dye laser, or solid state laser while the optoacoustic probes may include a piezodetector that is based on piezo-materials selected from piezopolymers and piezoceramics, capacitive micromachined ultrasonic transducers (CMUTs), and optically-based ultrasound detectors including interferometric detectors, optical beam deflecting detectors, pressure-sensitive optical elements.

Disclosed herein, in some aspects, is an apparatus for measurement of blood oxygenation of a subject or a blood vessel of the subject, the apparatus comprising: a) a base configured for placement against a portion of a body of a subject; and b) an adapter rotatably coupled to the base, the adapter comprising a housing configured to removably couple to one or more of an ultrasound probe or an optoacoustic probe such that said one or more of the ultrasound probe or optoacoustic probe is rotatable at a plurality of angles relative to the portion of the body.

In some embodiments, the apparatus further comprises an anchor coupled to the base, wherein the adapter is rotatably coupled to the anchor. In some embodiments, the apparatus further comprises a shaft extending from the adapter and at least partially through a cavity within the anchor, the shaft configured to rotate within the cavity. In some embodiments, the shaft rotates in concert with the adapter. In some embodiments, the shaft is detachably coupled with the adapter. In some embodiments, the anchor is detachably coupled with the base. In some embodiments, the apparatus further comprises a locking mechanism configured to lock a rotational position of the adapter relative to the base. In some embodiments, the locked rotational position of the adapter corresponds to a desired angle of the plurality of angles between the ultrasound probe, an optoacoustic probe, or both and the portion of the body. In some embodiments, the locking mechanism comprises a clamp that clamps the shaft with the anchor, such that the adapter is prevented from further rotation. In some embodiments, the ultrasound probe is coupled with an ultrasound probe extension. In some embodiments, the optoacoustic probe is coupled with an optoacoustic probe extension. In some embodiments, the adapter further comprises a proximal opening at the proximal end. In some embodiments, the proximal opening is configured to receive the ultrasound probe, the ultrasound probe extension, the optoacoustic probe, the optoacoustic probe extension, or a combination thereof. In some embodiments, at least a distal portion of the ultrasound probe, the ultrasound probe extension, the optoacoustic probe, the optoacoustic probe extension, or a combination thereof comprise the same form factor. In some embodiments, the base comprises an adhesive to attach to at least one portion of the body of the subject. In some embodiments, the adhesive is a two-sided adhesive. In some embodiments, the apparatus further comprises a second base coupled to a second anchor, the second base and second anchor coupled to the adapter via a second shaft. In some embodiments, the apparatus further comprises a first screw configured to clamp the shaft to the anchor, and a second screw configured to clamp the second shaft to the second anchor, thereby preventing further rotation by the adapter. In some embodiments, the portion of the body comprises a chest region and/or a neck region. In some embodiments, the portion of the body comprises an upper chest region and/or a lower neck region.

Disclosed herein, in some aspects, is a system for measurement of blood oxygenation of a subject or a blood vessel of the subject, the system comprising: a) an ultrasound probe; b) an optoacoustic probe; c) a system controller operatively coupled to the ultrasound probe, the optoacoustic probe, or both; d) an apparatus configured to align one or both of the ultrasound probe and optoacoustic probe with at least one portion of the body of the subject, the apparatus comprising: i) a base configured for placement against the at least one portion of the body of the subject; and ii) an adapter rotatably coupled to the base, the adapter comprising a housing configured to removably couple to one or more of the ultrasound probe or the optoacoustic probe, such that said one or more of the ultrasound probe or the optoacoustic probe is rotatable at a plurality of angles relative to the at least one portion of the body of the subject.

In some embodiments, the system further comprises an anchor coupled to the base, wherein the adapter is rotatably coupled to the anchor. In some embodiments, the system further comprises a shaft extending from the adapter and at least partially through a cavity within the anchor, the shaft configured to rotate within the cavity. In some embodiments, the shaft rotates in concert with the adapter. In some embodiments, the shaft is detachably coupled with the adapter. In some embodiments, the anchor is detachably coupled with the base. In some embodiments, the system further comprises a locking mechanism configured to lock a rotational position of the adapter relative to the base. In some embodiments, the locked rotational position of the adapter corresponds to a desired angle of the plurality of angles between the ultrasound probe, an optoacoustic probe, or both and the at least one portion of the body of the subject. In some embodiments, the locking mechanism comprises a clamp that clamps the shaft with the anchor, such that the adapter is prevented from further rotation. In some embodiments, the ultrasound probe is coupled with an ultrasound probe extension. In some embodiments, the optoacoustic probe is coupled with an optoacoustic probe extension. In some embodiments, the adapter further comprises a proximal opening at the proximal end. In some embodiments, the proximal opening is configured to receive the ultrasound probe, the ultrasound probe extension, the optoacoustic probe, the optoacoustic probe extension, or a combination thereof. In some embodiments, at least a distal portion of the ultrasound probe, the ultrasound probe extension, the optoacoustic probe, the optoacoustic probe extension, or a combination thereof comprise the same form factor. In some embodiments, the base comprises an adhesive to attach to at least one portion of the body of the subject. In some embodiments, the adhesive is a two-sided adhesive. In some embodiments, the system further comprises a second base coupled to a second anchor, the second base and second anchor coupled to the adapter via a second shaft. In some embodiments, the system further comprises a first screw configured to clamp the shaft to the anchor, and a second screw configured to clamp the second shaft to the second anchor, thereby preventing further rotation by the adapter. In some embodiments, the system further comprises a display interface operatively coupled to the system controller, the display interface configured to display an image captured by the ultrasound probe and/or blood oxygenation measurement data via the optoacoustic probe. In some embodiments, the system controller is configured to activate and/or deactivate the ultrasound probe and/or the optoacoustic probe. In some embodiments, the system controller is operatively coupled to a subject's interface. In some embodiments, one or more of the first portion of the body and the second portion of the body comprises a chest region and/or a neck region. In some embodiments, one or more of the first portion of the body and the second portion of the body comprises an upper chest region and/or a lower neck region.

Disclosed herein, in some aspects, is a method for measurement of blood oxygenation of a subject or a blood vessel of the subject, the method comprising: a) placing a base against a first portion of a body of a subject, wherein an adapter is rotatably coupled to the base; b) aligning an ultrasound probe with the first portion of the body or a second portion of the body via the adapter; c) adjusting an angle of alignment between the ultrasound probe and the first portion or the second portion of the body via rotation of the adapter to a first rotational position relative to the base, so as to identify a location of a blood vessel; d) based on an image obtained from the ultrasound probe, identifying the location of the blood vessel; e) aligning an optoacoustic probe at said angle of alignment with the first portion or the second portion, via the adapter that remains at the first rotational position; and f) measuring the blood oxygenation within the blood vessel with the optoacoustic probe.

In some embodiments, prior to step (d), based on an image obtained from the ultrasound probe that may not identify the location of the blood vessel, further adjusting the angle of alignment between the ultrasound probe and the first portion or the second portion of the body via rotation of the adapter to a second rotational position relative to the base. In some embodiments, prior to step (d), based on an image obtained from the ultrasound probe that may not show an optimum angle of alignment for optoacoustic measurement of blood oxygenation in the blood vessel, further adjusting the angle of alignment between the ultrasound probe and the first portion or the second portion of the body via rotation of the adapter to a second rotational position relative to the base. In some embodiments, prior to step (d), based on identifying no rotational positions of the adapter available to identify the blood vessel, moving the base to another location on the first or second portion of the body. In some embodiments, prior to step (d), based on identifying no rotational positions of the adapter available to obtain an optimum angle of alignment for optoacoustic measurement of blood oxygenation in the blood vessel, moving the base to another location on the first portion or second portion of the body. In some embodiments, prior to aligning the optoacoustic probe, the rotational position of the adapter is locked via a locking mechanism, so as to prevent further rotation of the adapter relative to the base. In some embodiments, the locking mechanism comprises a clamp that clamps the shaft with the anchor, such that the adapter is prevented from further rotation. In some embodiments, prior to aligning the optoacoustic probe, the base is secured to the first portion or the second portion of the body. In some embodiments, placing the base against the at least one portion of the body comprises adhering the base to the first portion of the body. In some embodiments, aligning the ultrasound probe comprises at least partially inserting the ultrasound probe within the adapter. In some embodiments, aligning the ultrasound probe comprises at least partially inserting the ultrasound probe into an ultrasound probe extension (UPE), and at least partially inserting the UPE within the adapter. In some embodiments, aligning the optoacoustic probe comprises removing the ultrasound probe and/or UPE from the adapter, and at least partially inserting the optoacoustic probe within the adapter. In some embodiments, aligning the optoacoustic probe comprises removing the ultrasound probe and/or UPE from the adapter, and at least partially inserting the optoacoustic probe into an optoacoustic probe extension (OPE), and at least partially inserting the OPE within the adapter. In some embodiments, the image is obtained via the ultrasound probe by using a system controller to activate said ultrasound probe. In some embodiments, the image is displayed on a display interface operatively coupled to the system controller. In some embodiments, measuring the blood oxygenation comprises using a system controller to activate said optoacoustic probe. In some embodiments, the method further comprises displaying the blood oxygenation of the blood vessel using a display interface operatively coupled to the system controller. In some embodiments, one or more of the first portion of the body and the second portion of the body comprises a chest region and/or a neck region. In some embodiments, one or more of the first portion of the body and the second portion of the body comprises an upper chest region and/or a lower neck region. In some embodiments, the blood vessel comprises a left innominate vein, a right innominate vein, a superior vena cava, an aorta, a right internal jugular vein, a left internal jugular vein, a left subclavian vein, a right subclavian vein, or a combination thereof.

Disclosed herein, in some aspects, is an apparatus for measurement of blood oxygenation of a subject or a blood vessel of the subject, the apparatus comprising: a base configured for placement against a portion of a body of a subject; and an adapter coupled to the base at a fixed angle relative to the portion of the body of the subject, the adapter comprising a housing configured to removably couple to one or more of an ultrasound probe or an optoacoustic probe such that said one or more of the ultrasound probe or optoacoustic probe is positioned in a predetermined and fixed angle relative to the portion of the body. In some embodiments, said apparatus is used for any system or method disclosed herein.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A shows an exemplary ultrasound image of the right internal jugular vein of a sheep with the optoacoustic probe was placed on the anterior neck surface and measured for optoacoustic oxygenation, per one or more embodiments herein;

FIG. 1B shows a graph of exemplary optoacoustic signal obtained from the IJV at the 10-11 mm point shown in FIG. 1A, per one or more embodiments herein;

FIG. 1C shows an exemplary graph of optoacoustic determination of venous oxygen at 82±2% versus 83% via co-oximetry [dashed line], per one or more embodiments herein;

FIG. 2 shows a diagram of certain aspects of the venous, arterial, and skeletal anatomy of the upper thorax with anatomical features, per one or more embodiments herein;

FIG. 3 shows a diagram of certain aspects of the venous, arterial, and skeletal anatomy of the upper thorax without anatomical features, per one or more embodiments herein;

FIG. 4A shows an exemplary image of U/S measurements made with a human subject supine with the head turned toward the left with a 13 MHz ultrasound (U/S) probe (GE Vivid) placed in the lateral to left supra sternal notch, per one or more embodiments herein;

FIG. 4B shows an exemplary image of a Pulse wave Doppler positioned on center of LIV and demonstrates a low frequency venous pulse waveform (5) that varied with respiration, per one or more embodiments herein;

FIG. 4C shows an exemplary image of placement of an optoacoustic detector over the sternal notch on a human subject, per one or more embodiments herein;

FIG. 5A shows a graph of exemplary optoacoustic signals for the LIV obtained with the prototype probe of FIG. 4C, per one or more embodiments herein;

FIG. 5B shows a graph of exemplary S_{LIV}O₂ measurements determined from averaging 20-30 optoacoustic signals over 3-4 min, per one or more embodiments herein;

FIG. 6A shows optoacoustic determination of venous oxygenation in same subject as FIGS. 5A-5B with blood oxygenation values but with a design allowing a closer approach under the clavicle, per one or more embodiments herein;

FIG. 6B shows a graph of an exemplary optoacoustic determination of venous oxygenation in same subject as FIGS. 5A-5B with optoacoustic signal with depth through the tissue with blood with a design allowing a closer approach under the clavicle, per one or more embodiments herein;

FIG. 7A shows an exemplary graph of optoacoustic determination of venous oxygenation in same subject as FIGS. 5A-5B and FIGS. 6A-6B but with a design allowing a closer approach under the clavicle as used in generating the data of FIGS. 6A-6B, per one or more embodiments herein;

FIG. 7B shows an exemplary graph of an optoacoustic signal with depth through the tissue, per one or more embodiments herein;

FIG. 8A shows a graph of an exemplary optoacoustic determination of venous oxygenation, with oxygenation values, in same subject as FIGS. 5A-5B, FIGS. 6A-6B, and FIGS. 7A-7B but with a design allowing a closer approach under the clavicle as used in generating the data of FIGS. 6A-6B and FIGS. 7A-7B, per one or more embodiments herein;

FIG. 8B shows a graph of an exemplary optoacoustic determination of venous oxygenation with depth through the tissue.in the same subject as FIGS. 5A-5B, FIGS. 6A-6B, and FIGS. 7A-7B but with a design allowing a closer approach under the clavicle as used in generating the data of FIGS. 6A-6B and FIGS. 7A-7B, per one or more embodiments herein;

FIG. 9A shows a side view image of an exemplary optoacoustic interface prototype, per one or more embodiments herein;

FIG. 9B shows an exemplary image of the small rectangular face of skin contact, per one or more embodiments herein;

FIG. 9C shows an image of an exemplary embodiment of an optoacoustic system with data display, per one or more embodiments herein;

FIG. 10 is a diagram of an exemplary protocol for sampling to confirm that sampling the left innominate vein (LIV) will correlate strongly with concurrent superior vena cava (SVC) oxygen saturation over an operative and perioperative course, per one or more embodiments herein;

FIG. 11 is a chart of hemorrhage classifications based on determinations of venous oxygenation, per one or more embodiments herein;

FIG. 12 shows an exemplary image of an optoacoustic probe, per one or more embodiments herein;

FIG. 13A shows an image of an exemplary holder that controls the positioning of both U/S and optoacoustic probes sequentially, per one or more embodiments herein;

FIG. 13B shows a top view and a side view image of an exemplary holder prototype, the geometry which allows for inserting ultrasound and optoacoustic probes, per one or more embodiments herein;

FIG. 13C shows a perspective image of the exemplary holder and optoacoustic probe of the holder shown in FIG. 13A, per one or more embodiments herein;

FIG. 13D shows a perspective image of an exemplary holder and a handle, per one or more embodiments herein;

FIG. 14A shows a first image of an exemplary combined ultrasound imaging and optoacoustic monitoring probe, per one or more embodiments herein;

FIG. 14B shows a second image of an exemplary combined ultrasound imaging and optoacoustic monitoring probe, per one or more embodiments herein;

FIG. 15A shows a first image of a combined ultrasound and optoacoustic monitoring probes wherein a Doppler ultrasound system is adapted combination with for optoacoustic monitoring, per one or more embodiments herein;

FIG. 15B shows a second image of a combined ultrasound and optoacoustic monitoring probes wherein a Doppler ultrasound system is adapted combination with for optoacoustic monitoring, per one or more embodiments herein;

FIG. 15C shows a third image of a combined ultrasound and optoacoustic monitoring probes wherein a Doppler ultrasound system is adapted combination with for optoacoustic monitoring, per one or more embodiments herein;

FIG. 15D show an exemplary optoacoustic signal obtained with the combined Doppler and optoacoustic device depicted in FIG. 15A, per one or more embodiments herein;

FIG. 15E shows a graph of exemplary blood oxygenation values obtained with the device depicted in FIG. 15A, per one or more embodiments herein;

FIG. 16A show a side cross-sectioned view of an exemplary dual mount Doppler ultrasound guidance and optoacoustic measurement apparatus, per one or more embodiments herein;

FIG. 16B show a perspective cross-sectioned view of an exemplary dual mount Doppler ultrasound guidance and optoacoustic measurement apparatus, per one or more embodiments herein;

FIG. 16C shows perspective bottom view of an exemplary dual mount Doppler ultrasound guidance and optoacoustic measurement apparatus, per one or more embodiments herein;

FIG. 17 shows images of an exemplary dual ultrasound (or Doppler) probe and an optoacoustic probe, per one or more embodiments herein;

FIG. 18 shows a perspective view image of an exemplary apparatus for ultrasound guided optoacoustic blood oxygenation measurement with extension components for the ultrasound probe and optoacoustic probe, per one or more embodiments herein;

FIG. 19 shows a perspective view image of an exemplary extension for an ultrasound probe inserted in the apparatus from FIG. 18, per one or more embodiments herein;

FIG. 20 shows a top view image of the exemplary extension and apparatus from FIG. 19, per one or more embodiments herein;

FIG. 21 shows an image of a front view of an upper chest of a subject, including an exemplary location for placing the apparatus from FIGS. 18-20 to align an ultrasound probe and optoacoustic probe with a target blood vessel, per one or more embodiments herein;

FIG. 22 shows a perspective view image of an exemplary system for ultrasound guided optoacoustic blood oxygenation measurement comprising a system controller, an apparatus, and an optoacoustic probe, per one or more embodiments herein;

FIG. 23 shows a perspective view image of the exemplary apparatus and optoacoustic probe from FIG. 22, per one or more embodiments herein;

FIG. 24 shows a perspective view image of the exemplary apparatus from FIG. 22 with an ultrasound probe inserted into an extension that is inserted into an adapter of the apparatus, per one or more embodiments herein;

FIG. 25 shows an exploded view image of an exemplary apparatus and ultrasound probe from FIG. 24 with the ultrasound probe, extension, and apparatus separated, per one or more embodiments herein;

FIG. 26 shows a front view image of an exemplary apparatus ultrasound guided optoacoustic blood oxygenation measurement, per one or more embodiments herein; and

FIG. 27 shows a flowchart for an exemplary method for ultrasound guided optoacoustic blood oxygenation measurement, according to many embodiments herein.

### DETAILED DESCRIPTION OF THE INVENTION

Provided herein is a unique, noninvasive method of optoacoustic measurement of SO₂ in major veins to facilitate rapid diagnosis and treatment of circulatory shock with and without TBI. Venous oxygen (hemoglobin) saturation (SO₂) is a single, easily interpreted number that represents systemic and local factors that influence systemic oxygen delivery (DO₂) and oxygen consumption (VO₂). However, existing assessment of venous saturation is invasive. Presently such measurements must be made by continuous oximetric catheters for the pulmonary artery (“PA”) (providing mixed venous SO₂), jugular bulb (providing brain SO₂) or the superior vena cava (“SVC”), and require invasive catheterization, which is risky and consumptive.

In certain embodiments, measurement of SO₂ in the left innominate vein (S_{LIV}O₂) provides rapid diagnosis and treatment of circulatory shock with and without TBI. The left innominate vein, also known as the brachiocephalic vein, collects venous blood from the jugular vein and is the main venous tributary for the superior vena cava and is thus a primary vessel for measuring and monitoring SO₂ in the venous brain drainage. The noninvasive optoacoustic measurement of S_{LIV}O₂ disclosed herein provides rapid recognition of occult hemorrhagic shock and subsequently provides resuscitation monitoring such that over-resuscitation is less likely. The technology is particularly valuable during prolonged field care, while awaiting evacuation. Based upon evidence of continued or previously unrecognized hemorrhage enabled by the technology disclosed herein, a combat or civilian medic could initiate fluid resuscitation to maintain adequate perfusion during the interval before definitive control of hemorrhage may be achieved.

In certain embodiments, algorithms for use of S_{LIV}O₂ data generated using the methods and apparatus disclosed herein will resemble those used with invasive SVC oximetry. For instance, Rivers et al. used a threshold of <70% saturation to define the need for interventions such as fluid or blood administration or inotropic infusions. See Rivers, et al. supra. Low S_{LIV}O₂ may be used in exactly the same way, with the exception that S_{LIV}O₂ may be measured noninvasively within one minute even during ambulance or helicopter transport, whereas central venous oximetry requires central venous catheterization. S_{LIV}O₂ monitoring is also easily incorporated into automated decision-support or closed-loop management systems as these evolve for use in civilian trauma subjects.

Ultrasound guided optoacoustic monitoring is expected to provide valuable diagnostic information for many clinical applications. One of them is optoacoustic monitoring of blood variables as such as blood oxygenation in blood vessels and in tissues. Ultrasound-guided optoacoustic monitoring of central venous oxygenation may be used for early diagnosis and management of circulatory shock (including that induced by hemorrhage). Either standard ultrasound imaging or Doppler technology, or both may be used for guidance of optoacoustic probe to perform targeted probing of specific blood vessels and measurement of blood oxygenation. Ultrasound guidance to locate the large vein for oxygenation may be performed in a number of modes including:

In certain embodiments provided herein, methods and apparatus for optoacoustics for measurement of blood oxygenation in the innominate vein. Ultrasound imaging and Doppler techniques provide important information on location of this blood vessel.

While the making and using of various embodiments of the present invention are discussed in detail below, it should be appreciated that the present invention provides many applicable inventive concepts which may be employed in a wide variety of specific contexts. The specific embodiment discussed herein are merely illustrative of specific ways to make and use the invention and do not delimit the scope of the invention.

### TERMS AND DEFINITIONS

The following abbreviations are used throughout this application:
BV Basilic Vein
DO₂ Oxygen Delivery
IJV Internal Jugular Vein
LIV Left Innominate Vein
NIR Near-Infrared
PA Pulmonary Artery
PEEP Positive End-Expiratory Pressure
PFC Prolonged Field Care
PLD Pulsed Laser Diodes
PZT Piezoceramic lead zirconate titanate (Pb[Zr (x)Ti(1−x)]O₃
PVDF piezopolymer polyvinylidene fluoride
S_{LIV}O₂ Oxygen Saturation measured at the LIV
SO₂ Venous hemoglobin or oxygen saturation
SPAO₂ mixed venous oxygen saturation by invasive pulmonary artery catheterization
SSSsO₂ Oxygen saturation measured at the Superior Sagittal Sinus
SVC Superior Vena Cava
SvO₂, mixed venous saturation
TBI Traumatic Brain Injury
U/S Ultrasound
VO₂ oxygen consumption

To facilitate the understanding of this invention, and for the avoidance of doubt in construing the claims herein, a number of terms are defined below. Terms defined herein have meanings as commonly understood by a person of ordinary skill in the areas relevant to the present invention.

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” in some cases refers to an amount that is approximately the stated amount.

As used herein, the term “about” refers to an amount that is near the stated amount by 10%, 5%, or 1%, including increments therein.

As used herein, the term “about” in reference to a percentage refers to an amount that is greater or less the stated percentage by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. The terms “comprising” (and any form thereof such as “comprise” and “comprises”), “having” (and any form thereof such as “have” and “has”), “including” (and any form thereof such as “includes” and “include”) or “containing” (and any form thereof such as “contains” and “contain”) are inclusive or open-ended and do not exclude additional, unrecited elements or method steps.

The term “effective” as used in the specification and claims, means adequate to provide or accomplish a desired, expected, or intended result. The terms “about” or “approximately” are defined as being close to as understood by one of ordinary skill in the art, and in one non-limiting embodiment the terms are defined to be within 10%, within 5%, within 1%, and in certain aspects within 0.5%.

Provided herein is a non-invasive, highly portable, optoacoustic apparatus that rapidly assesses SO₂, in the innominate vein, which closely approximates SsvcO₂. The monitor transmits short-duration pulses of near-infrared (NIR) light, which are absorbed by oxygenated and deoxygenated Hgb and subsequently generate ultrasound signals that accurately measure SO₂. This novel optoacoustic technology could obviate the need for central venous catheterization, while accurately and frequently assessing central venous SO₂ for casualties in shock or at risk for shock.

Optoacoustic technology: Laser optoacoustic imaging techniques combine the merits of optical tomography (high optical contrast) and ultrasound imaging (insignificant scattering of acoustic waves) to yield a noninvasive diagnostic modality with high contrast, sensitivity, and resolution. The high resolution, sensitivity and contrast of optoacoustic techniques provide monitoring of total Hgb concentration, oxygenated Hgb, deoxygenated Hgb and, depending on the wavelengths used, carboxyHgb and metHgb with excellent accuracy, specificity, and sensitivity. Laser optoacoustics, recently developed as a technique for tissue characterization and diagnostic imaging, provides continuous, noninvasive, highly accurate measurement. Optoacoustic techniques utilize sensitive detection of laser-induced ultrasonic waves rather than optical signals. Because the acoustic waves travel in a straight line from the source, the depth of the target blood vessel may be precisely calculated from the time required for the signal to return and the speed of sound through tissue. Transmission of ultrasound signals in a straight line differentiates optoacoustic measurements from pure optical measurements, in which returning optical signals are scattered, as is the incident light. Time-resolved detection of the pressure profiles by ultrasound transducers and analysis of the pressure signals facilitate high-resolution reconstruction of optoacoustic images. Optoacoustic techniques may pinpoint structures in optically turbid and opaque tissues at depths as great as eight centimeters with spatial resolution≤0.5 millimeters and to reconstruct optoacoustic images.

OxyHgb and de-oxyHgb have high absorption coefficients in the visible and NIR spectral range. Therefore, both the amplitude and spatial distribution of the generated optoacoustic pressure induced in blood are dependent on the Hgb saturation and concentration (calculated as oxyHgb÷total Hgb). High z-axial resolution of the optoacoustic technique permits direct measurement of Hgb saturation in large blood vessels because the optoacoustic waves induced in blood arrive at the acoustic transducer at a time that is directly proportional to the speed of sound in tissue. Since the Hgb absorption coefficient is dependent on Hgb SO₂, laser sources with wavelengths of approximately 805 nm (isosbestic point where oxyHgb and deoxyHgb have equal absorption) for are utilized for Hgb monitoring; and then, using the obtained [Hgb] value, wavelengths of approximately 1064 nm are used for oxygenation monitoring because oxyHgb and deoxyHgb have strong differences in absorption. Thus, by analyzing the temporal profile of optoacoustic pressure induced in blood by pulsed laser NIR light of various wavelengths, the absolute value of Hgb SO₂ may accurately be obtained.

In some embodiments, the emitted light is within the low end of the NIR spectral range, such as approximately 600 to 1300 nm, for example 760 nm, 800 nm, and 1064 nm. Such a wavelength range may result in deep penetration of the NIR radiation, which is sufficient for optoacoustic monitoring of hemoglobin saturation. The amount of laser energy applied for monitoring may be sufficiently small to prevent any thermal or mechanical damage to a subject's skin or a subject's or operator's ocular tissues because laser fluence levels are well below the maximum permissible exposures (MPE) for ocular tissues. In some embodiments, the laser energy is delivered at a power of approximately 1 μJ to 1 mJ.

Oxyhemoglobin and deoxyhemoglobin have high absorption coefficients in the visible and NIR spectral range. Therefore, both the amplitude and spatial distribution of the generated optoacoustic pressure induced in blood are generally dependent on total hemoglobin concentration [THb] and hemoglobin saturation (calculated as oxyhemoglobin/[THb]). The high resolution of the disclosed measurement technique enables direct measurement of [THb] and saturation in large blood vessels. In some embodiments, saturation may be assessed using an optical parametric oscillator (OPO) pumped by Nd-YAG laser to generate five important wavelengths: 800 or 805 nm (isosbestic point where oxy- and deoxyhemoglobin have equal absorption) and 700, 730, 760 nm, and 1064 nm, which are wavelengths at which oxy- and deoxy-hemoglobin have strong differences in absorption. In some embodiments, the concentration of different molecules may be of interest such that other wavelengths are chosen.

As previously mentioned, the acoustic signal generally returns in a straight line from the target. Laser optoacoustic imaging techniques combine the merits of optical tomography (high optical contrast) and ultrasound imaging (minimal scattering of acoustic waves) to yield a noninvasive diagnostic modality with high contrast, sensitivity, and resolution. The high resolution, sensitivity, and contrast of optoacoustic techniques provide monitoring of [THb], oxygenated and deoxygenated hemoglobin with excellent accuracy, specificity, and sensitivity. Transmission of ultrasound signals in a straight line differentiates optoacoustic measurements from pure optical techniques in which both incident and returning optical signals are scattered by passage through tissue. Optoacoustic imaging may visualize structures in optically turbid and opaque tissues at depths as great as several centimeters with a spatial resolution≤0.5 mm and may reconstruct optoacoustic images. In summary, the merits of optoacoustic monitoring include, but are not limited to: (1) noninvasiveness, (2) accurate, quantitative measurements, (3) continuous, real-time monitoring, (4) high spatial resolution, and (5) compact dimensions.

In clinical optoacoustic monitoring of SO₂ in the innominate vein, the acoustic detector will monitor signals that return toward the optical source (backward mode). Merits of optoacoustic monitoring include: 1) noninvasiveness, 2) accurate, quantitative measurements, 3) continuous, real-time monitoring, 4) high spatial resolution, 5) compact dimensions. In certain embodiments the system is miniaturized to operate from a device the size of or smaller than a notebook computer thus permitting wide application of the sensor at all echelons of care.

The following illustrative examples are representative of embodiments of the software applications, systems, and methods described herein and are not meant to be limiting in any way.

### Example 1—Comparative Studies of Venous SO₂ in the Basilic Vein (BV) and Internal Jugular Vein (IJV)

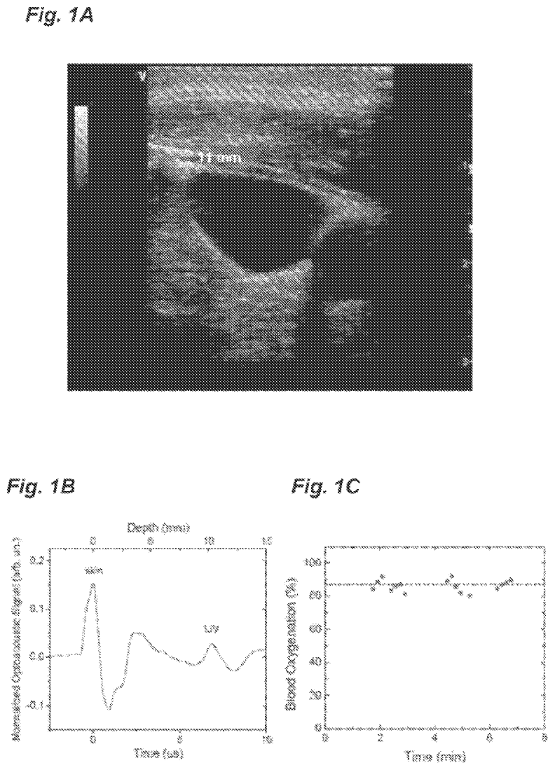

In vivo tests in large animals (sheep) demonstrated that a prototype system measures SO₂ accurately and precisely (correlation: r2 0.99; bias=2.47%; SD=±2.3%) in comparison to the gold standard hemoximetry. In a previous study, a dual-probe optoacoustic prototype was designed to detect and compare venous SO₂ in the basilic vein (BV) and internal jugular vein (IJV) by both a validation study and a clinical concept testing. The validation study focused on the IJV oxygenation comparison between optoacoustic and the gold standard hemoximetry. See Petrov I Y, et al Optoacoustic measurement of central venous oxygenation for assessment of circulatory shock: clinical study in cardiac surgery subjects. Proc. SPIE 8943 (89430Y) (2014) 1-5. In brief, the IJV was interrogated by ultrasound (U/S), depth was recorded, and the skin was marked for IJV borders. FIG. 1A shows an ultrasound image of the right internal jugular vein of a sheep. The optoacoustic probe was placed on the anterior neck surface and measured for optoacoustic oxygenation. FIG. 1B shows the optoacoustic signal obtained from the IJV at the 10-11 mm point shown in FIG. 1A. A central line was placed and confirmation hemoximetry from IJV was obtained using the finder needle. FIG. 1C shows optoacoustic determination of venous oxygen 82±2% versus 83% via co-oximetry [dashed line]. Data showed that the depth calculated by U/S and optoacoustics for IJV were ±1.7 mm and the venous oxygen saturation SO₂ measurements for comparing hemoximetry to optoacoustic were 3±2%, demonstrating high accuracy. Thus, comparing gold-standard measurements for IJV oxygenation and signal acquisition depth may accurately be obtained by the optoacoustic prototype.

### Example 2—Initial Development of S_{LIV}O₂ Measurement

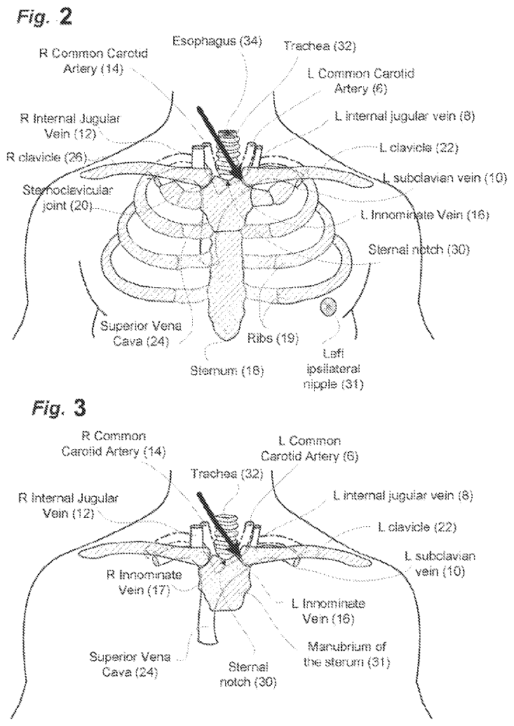

In efforts to improve SO₂ measurements for identifying and monitoring shock, several important improvements were made. First, signal stability was achieved through use of a laser diode-based optoacoustic system with a high pulse repetition rate. This facilitated rapid measurements in real-time. Next, an ultrasound and optoacoustic window is identified to interrogate the left innominate vein (LIV) through the supra-sternal notch. Certain of the venous, arterial, and skeletal anatomy of the upper thorax is shown in FIG. 2 and FIG. 3 which show the left clavicle (22) and the right clavicle (26) and the connecting central, upper manubrium (31) of the sternum (18) also showing attachment of the upper ribs (19) to sternum (18). At the top center of the manubrium lies the suprasternal (aka sternal) notch (30), which overlies the left innominate vein (“LIV”) (16) and the right innominate vein (“RIV”) (17) and their connection to the superior vena cava (“SVC”) (24). The placement of manubrium (31) in relation to the trachea (32) is shown in FIG. 3, which eliminates certain features of FIG. 2 in order to more clearly depict the location of LIV (16) in relation to suprasternal notch (30). The heavy arrow shows the acoustic window for LIV (16), which is lateral and to the left of the suprasternal notch at a depth of 1-3 cm.

Anatomically, LIV (16) forms behind the left clavicle (22) and drains the left internal jugular vein (“IJV”) (8) and the left subclavian vein (10). The LIV is easier to access than RIV (17), which is more fully behind sternum (18). Both LIV (16) and MV (17) drain into SVC (24). Thus, the LIV approximates central venous oxygenation (SCVO₂), which has shown to be a superior endpoint in resuscitation from shock. Confirmation of whether the LIV oxygenation (S_{LIV}O₂) obtained by invasive catheterization is comparable to SCVO₂ in a broad population of subjects as well as mixed venous oxygen saturation by invasive pulmonary artery catheterization (SPAO₂=mixed venous saturation=S vO₂) will be obtained from clinical studies.

In one embodiment, an optoacoustic oxygenation monitoring system is provided to improve methods for resuscitation. Guiding resuscitative efforts based on S_{LIV}O₂, will better stabilize casualties with shock and TBI and provide an early detection of life-threatening injuries. Complimentary measures of brain venous oxygenation, such as S_{LIV}O₂, or SSSsO₂ could help mitigate progressive brain injury. In one embodiment, SO₂ indices are incorporated into a decision support or autonomous platform that would include resuscitation limits, need for blood or other vaso-actives. S_{LIV}O₂, does not suffer limitations by current perfusion assessment in field such as capnometry. Specifically, S_{LIV}O₂ does not require intubation and accuracy is not limited by anatomic and physiologic deadspace.

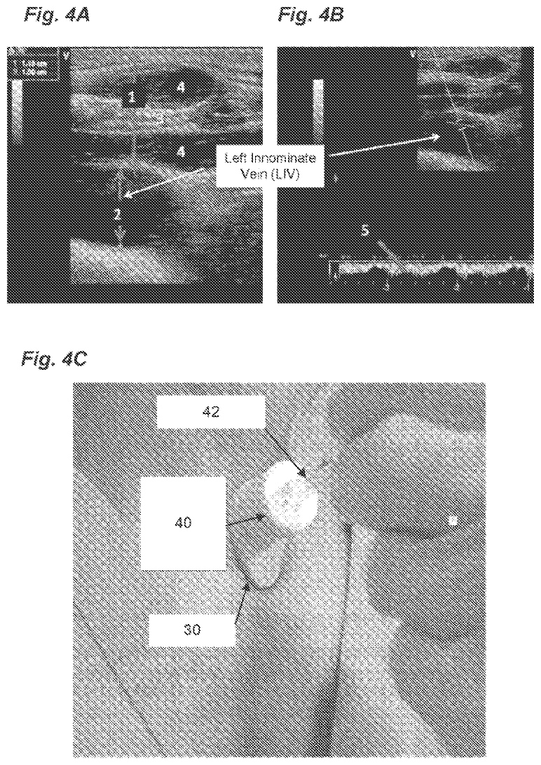

Optoacoustic determination of LIV access: Volunteers (n=5) were recruited to determine if an acoustic window for ultrasound and optoacoustic signals may be obtained for the innominate veins and in particular the LIV. Demographics of the volunteers were broad including: ages 24-70 yr., Ht. 160-195 cm, Wt. 50-119 kg and gender (4 males and 1 female). 2D and Doppler ultrasound (“U/S”) were used to characterize the LIV in relation to the sternal notch. In brief, U/S (FIGS. 4A and 4B) and optoacoustic (FIG. 4C) measurements were made with the subject supine with the head turned toward the left. An acoustic U/S window for LIV was found 1-3 cm lateral to the left suprasternal notch with a 12 MHz probe (12L, General Electric, Milwaukee, Wis.) tilted 120-150° from skin surface and aimed towards the ipsilateral nipple. LIV was confirmed by 2D ultrasound (FIG. 4A) and Doppler waveform (FIG. 4B), both showing the LIV. To obtain the image in FIG. 4A, a 12 MHz ultrasound (U/S) probe (GE ultrasound imaging probe i1 2L-RS connected to a GE Vivid system) was placed in the lateral to left supra sternal notch. 2D image acquisition shows the left innominate vein (LIV) indicated by white arrow. The depth from skin surface to LIV (arrow 1) was 11.3 mm. LIV had a diameter of 10 mm (arrow 2). Connective tissue (3) and small muscle bands (4) also observed. FIG. 4B shows Pulse wave Doppler positioned on center of LIV and demonstrates a low frequency venous pulse waveform (5) that varied with respiration.

The depth of the LIV by U/S [mean±SEM] was 10.3±0.8 mm. As depicted in FIG. 4C, after locating the LIV by ultrasound, an optoacoustic prototype probe was placed in a similar direction and plane. The probe housing 40, includes an internal optical fiber and acoustic transducer element, had a similar profile as the ultrasound probe including a flat, high surface area contact with skin. Probe housing 40 was easily maintained in a stable position on the subject by virtue of positional handle 42 affixed to housing 40.

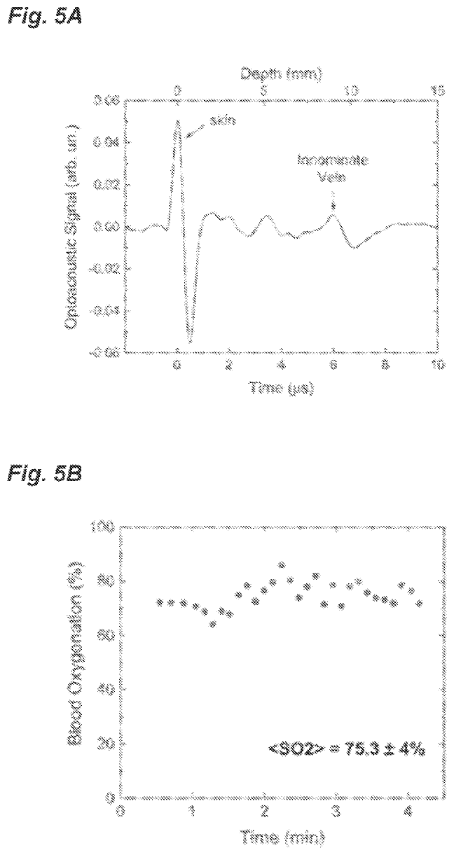

Oxygenation measurements of the LIV using optoacoustic prototype were confirmed by peak chromophore absorption signal at a depth consistent with ultrasound (9-10 mm below skin) (FIG. 5A). Once the absorption signal was obtained, S_{LIV}O₂ was determined from averaging 20-30 optoacoustic signals over 3-4 min (FIG. 5B) representative venous oxygenation from left innominate vein. Optoacoustic signal identification in FIG. 5A includes, skin, soft tissue [next peak] and innominate (LIV) based peak chromophore signal for LIV and depth.

Table 1 shows the range of S_{LIV}O₂ obtained in the 5 subjects. The mean±SEM for these subjects was 75±3%, which is similar to values in health for central venous oxygen saturation.

TABLE 1
Optoacoustic (“OA”) vs Ultrasound U/S Depth
and Oxygenation Determination in 5 Subjects

| Subject # | Depth: U/S (mm) | Depth: OA (mm) | <S_{LIV}O_{2>} % |
|---|---|---|---|
| CSIV101 | 11.3 | 9 | 73.6 |
| CSIV102 | 10.3 | 11.4 | 73.7 |
| CSIV103 | 11 | 8.7 | 82.7 |
| CSIV104 | 11.8 | 9.3 | 75.3 |
| CSIV105 | 7.2 | 6 | 71.3 |

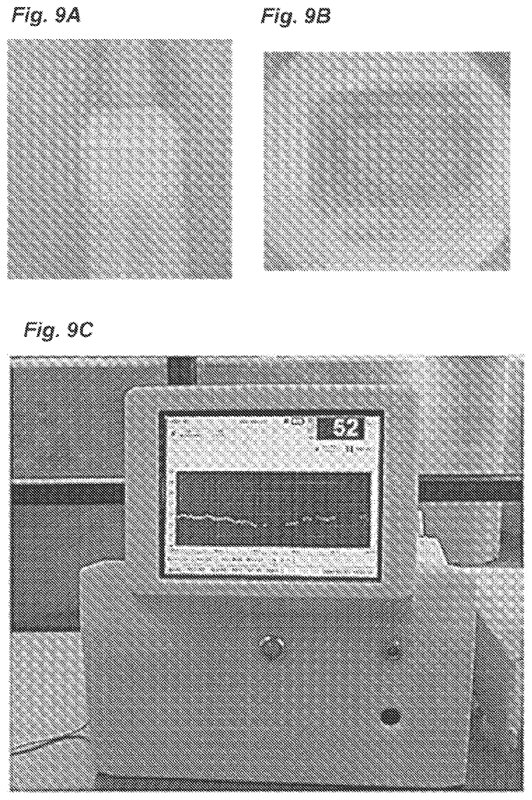

Of note, the depth measurements for optoacoustics (“OA”) were slightly lower (8.9±0.8 mm) in most cases compared to U/S (10.3±0.2 mm) due to the small amount skin displacement that is needed for the optoacoustic to skin coupling. While oxygenation data from the LIV could be obtained, the signal acquisition had some degree of variability. This was likely due to the optoacoustic probe's wide profile, rendering signal acquisition below the bend of the clavicle difficult. To address this issue, a new interface was prototyped as shown in FIGS. 9A and 9B. Specifically, the probe's face was elongated and narrowed to facilitate a more direct acoustic window as compared with the prototype of FIG. 4C. The design change allowed enhanced LIV interrogation. Additionally, since the LIV and probe alignment was improved, signal stability was augmented.

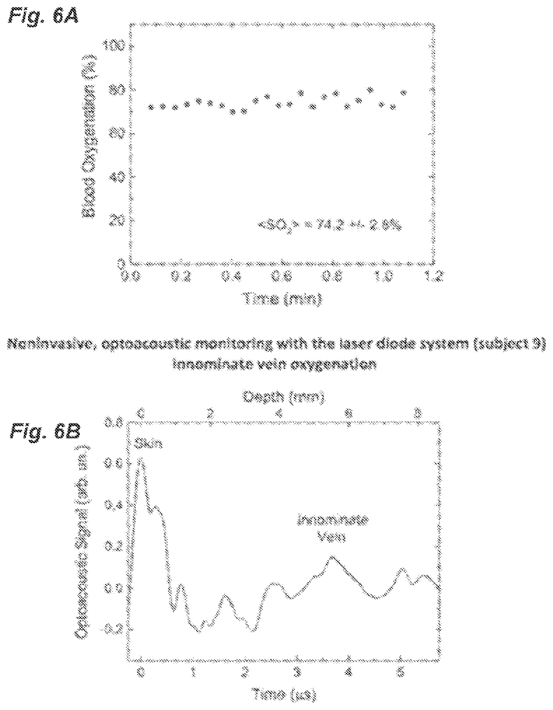

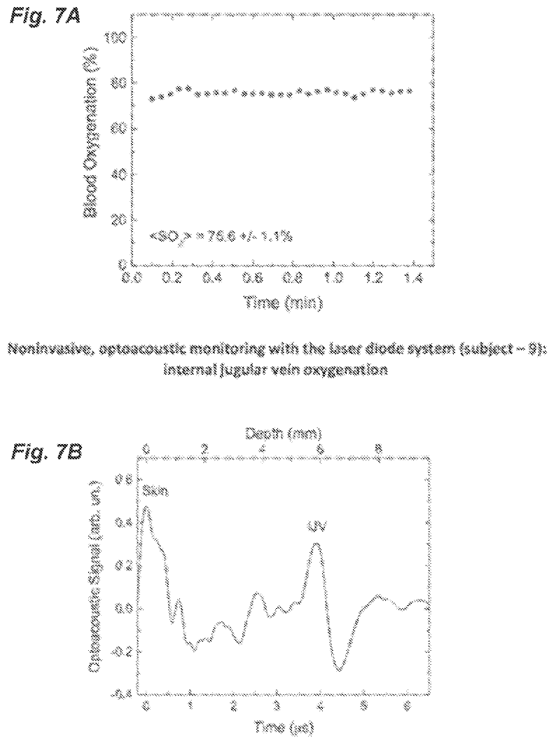

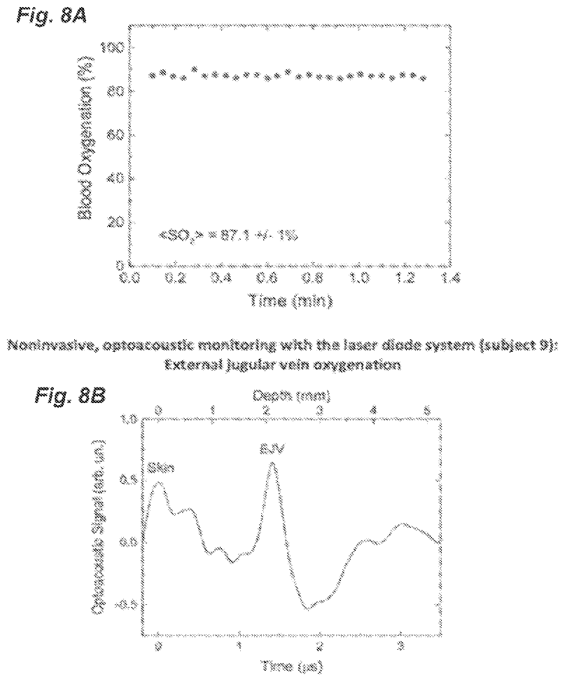

Using this design, optoacoustic oxygen saturation from left innominate vein (LIV), internal jugular vein (IJV) and external jugular vein, were determined in the same volunteer as the data depicted in FIGS. 5A and 5B. The probe interface allowed for greater contact with the skin surface. When the probe was placed under the clavicle and directed in a downward plane towards the left ipsilateral nipple (31) in FIG. 3, a substantially greater tissue displacement (5-7 mm) was observed compared to previous measurements using the flatter probe (average of 2 mm: Table 1). The amount of displacement was confirmed firstly by measuring the coverage from the tip of probe to the exposed portion of the probe from the overlying clavicle surface skin. Secondly, the distance using 2D ultrasound from the skin surface to the left innominate vein was measured and subtracted from the optoacoustic derived peak signal from the LIV. As indicated, the depth determined by ultrasound and optoacoustics differ considerably with this new probe design. It was observed that the greatest difference was with optoacoustic innominate vein measurement for the new probe designed to fit under the clavicle. Additionally, the innominate vein is not a compressible structure from external tissue displacement. On the other hand, excess displacement of the probe over the IJV results in compression and loss of venous signal. Therefore, there is a limit on the amount of force that may be applied over the IJV. It should be noted that despite several mm of tissue displacement by the probe, it does not produce subject discomfort. Thus, optoacoustic innominate vein depth and SO₂ determination with this probe design will likely result in a closer signal from skin surface than the IJV in post subjects. Having the peak chromophore signal closer to transducer confers the advantage of less scattering and greater signal stability, which likely explains the reduced signal variability. FIGS. 6A-6B, 7A-7B and 8A-8B show optoacoustic determination of venous oxygenation in same subject as FIGS. 5A and 5B. FIGS. 6A-6B show data for the left innominate vein with FIG. 6A showing blood oxygenation values and FIG. 6B showing the optoacoustic signal with depth through the tissue. FIGS. 7A-7B show data for the internal jugular vein with FIG. 7A showing blood oxygenation values and FIG. 7B showing the optoacoustic signal with depth through the tissue. FIGS. 8A-8B show data for the external jugular vein with FIG. 8A showing blood oxygenation values and FIG. 8B showing the optoacoustic signal with depth through the tissue. Oxygenation signal stability shows marked improvement.

The preliminary data demonstrate that venous SO₂, from a variety of venous sources, may be obtained using non-invasive, real-time, optoacoustic monitoring. Optoacoustic determination of venous oxygenation over the LIV, which lies beneath the left clavicular head, is an innovative approach to rapidly and non-invasively assess central venous oxygen saturation. The same technology platform may also be used to determine brain oxygenation including for initial TBI assessment and for monitoring brain oxygenation during Prolonged Field Care (“PFC”). Oxygenation in the venous effluents for internal jugular vein (SvO₂) and superior sagittal sinus (SSSSO₂) have been measured.

### Example 3—Noninvasive Optoacoustic Measurement of S_{LIV}O₂ to Permit Rapid Recognition of Shock

In one embodiment methods and apparatus are provided for noninvasive optoacoustic measurement of S_{LIV}O₂ to permit rapid recognition of shock and to subsequently provide robust resuscitation monitoring so that under and over-resuscitation do not occur. In certain cases, noninvasive monitoring of S_{LIV}O₂ is complemented by determining brain oxygenation with SuvO₂ or superior sagittal sinus (SSSSO₂) for TBI assessment. Optoacoustic determination of venous oxygenation may also be used as an adjunct monitor to prevent excessive PEEP, need for blood transfusion and other seamless adaptations for prolonged field care that includes: optimizing PEEP [SpO₂ vs S_{LIV}O₂], vital fluid choices [need for blood transfusion vs other fluids] and reducing oxygen consumption needs [fever, shivering thermogenesis vs need for paralysis fluid and sedation and anesthesia].

In one embodiment, a clinical validation protocol is used to establish efficacy of a device and method for ultrasound guided optoacoustic monitoring of oxygen saturation. In one such embodiments, cardiac surgical subjects are tested by comparing hemoximeter-derived oxygen saturation to noninvasive optoacoustic saturation. A pulmonary artery (PA) catheter is placed via left internal jugular introducer sheath. Each subject receives a series of optoacoustic and hemoximetry measurements. In one embodiment, validation is obtained that that LIV is equivalent to SVC oxygenation in a large population of subjects: Blood samples from an introducer inserted through the left internal jugular vein into the LIV is compared to proximal port (SVC) samples. In certain embodiments, physiologic validation is be obtained by comparing optoacoustic S_{LIV}O₂ to hemoximetry LIV in cardiac subjects during different physiologic states e.g., pre-surgery, three ICU time points, and discharge.

In certain embodiments, physiologic validation of optoacoustic S_{LIV}O₂ versus hemoximetry LIV is conducted. For each subject, data is collected including the type of surgery, duration of surgery and duration of pump run. Concurrent diseases and treatments may also be recorded, including blood, fluid, and inotrope/vasopressor infusion. In addition, demographic data is collected including gender, age, ethnicity, ejection fraction and other cardiac abnormalities. Sub-analysis is then performed using logistic regression to determine if any of these factors influence the optoacoustic measurements. Comparisons will be made from cardiac subjects during different physiologic states that occur during the perioperative period e.g., pre-surgery, OR post-surgery and three ICU time points.

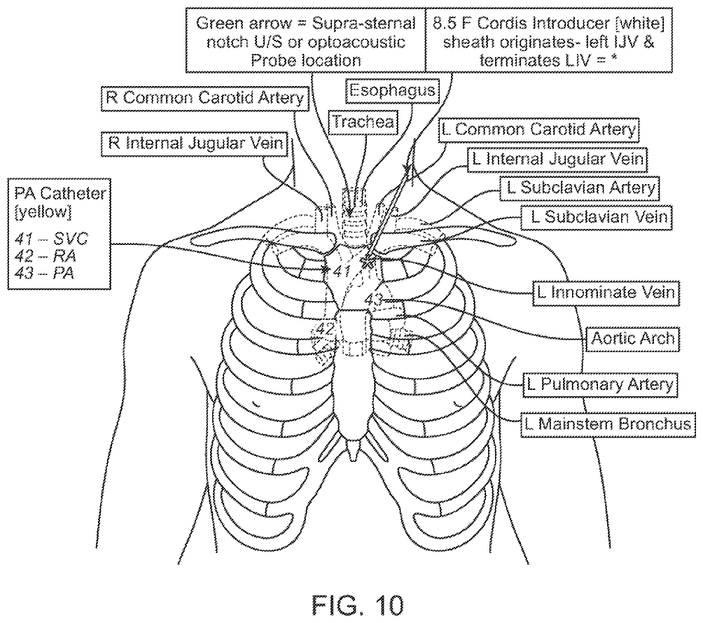

Validation of venous oxygenation equivalence is undertaken in certain embodiments. Pulmonary artery catheterization and monitoring are the standard of care for cardiac surgery. In one embodiment, equivalence testing could include placement of an introducer sheath (such as for example an 8.5 French Cordis or similar introducer sheath) into the left internal jugular by the anesthesiologist in an operating room under general anesthesia or sedation. A pulmonary artery (“PA”) catheter (such as for example an Edwards LifeScience PA catheter or the like) is then placed with the tip of the catheter located in the pulmonary artery and confirmed by PA occlusive waveform. One approach for comparing oxygenation measurements is shown in FIG. 10. The PA catheter has three ports that include the infusion port (super vena cava), proximal port (right atrium) and distal port (pulmonary artery).

Three consoles were designed and built that contained an optical parametric oscillator (OPO) as a laser source; a touchscreen, medical grade computer; power supplies and other control equipment. A fourth prototype system was built for measuring brain saturation in subjects with TBI. FIG. 9C depicts one such prototype of a display for an optoacoustic monitoring system initially designed for monitoring sagittal sinus saturation through the intact skull in subjects with TBI. This system may be markedly reduced in size for interrogating the left innominate or internal jugular veins since small laser power is only required to penetrate soft tissue. In one embodiment a system for prolonged field care could weigh less than or equal to 2.0 kg.

This prototype used pulsed laser diodes (PLD) stacks that have a higher repetition rate (1000 Hz) but utilize essentially the same control software and connecting cables. The higher pulse repetition frequency substantially reduces motion artifact vulnerability. Further, the PLD prototype has a smaller footprint and is therefore portable. In certain embodiments the diode-based system is miniaturized to a fraction of its current size, estimated to be ˜2.0 kg, because much less power is required to penetrate a few cm of soft tissue above venous structures. In vitro testing shows that a measurement of venous saturation may be completed within 30 seconds and the system may continuously update measurements every 1-3 seconds using a PLD system. In certain embodiments, the system provides for acquisition of more signals per second therefore reduce scanning time.

There are specific peak signals that are associated with depth, vessel size and chromophore characteristics. In certain embodiments, a novel peak signal recognition program may be employed that automatically identifies signals originating in clinically relevant veins, including the IJV, LIV, EJV, SCV, femoral (FV) and BV veins. This is similar to machine learning. The automated software may choose the largest signal from the detector when best positioned and convert those signals into quantitative saturation data. Software that digitizes and filters out background signals may also be used to enhance signal architecture. For example, when the probe is placed over the suprasternal notch and aimed left towards the left nipple various tissues are present e.g., connective tissue, small muscle bands and LIV. On the other hand, when pointing toward the right nipple, which has similar tissues but is over the right innominate vein, since it is in a deeper field, the homologous location may be used to subtract out or filter these signals.

In certain embodiments, further clinical validation protocols are used to establish efficacy of a device and method for ultrasound guided optoacoustic monitoring of oxygen saturation. Studies on volunteers generates requisite anatomic and trajectory data that define the anatomy of the acoustic window over the target vein including one or more of the IJV, LIV, EJV, SCV, femoral (FV) and BV veins.

In one embodiment of a clinical validation protocol used to establish efficacy of a device and method for ultrasound guided optoacoustic monitoring of oxygen saturation, the LIV is targeted and the optoacoustic trajectory for optimizing oxygenation signals is obtained. In one embodiment, volunteers are placed in supine in a Trendelenburg position. For each subject, measurements are made via ultrasound: distance from skin surface to vessel surface and midpoint, innominate vessel diameter, velocity profile by pulse wave Doppler and color flow mapping. An ultrasound probe is placed in the suprasternal notch and aimed at the left ipsilateral nipple until the innominate vein is found. The angle and direction of the ultrasound probe in relation the body in two different axes, at which the innominate vein is best interrogated, will be measured using an adjustable protractor arm. The two axes include: caudad to cephalad and medial to lateral. After the ultrasound measurements are complete, the optoacoustic probe is used to measure innominate vein oxygen saturation. In certain embodiments an iterative probe interface is used. An ultrasound probe is first applied to the subject and the vessel of interest is located. The interface is left in place stably affixed to the subject and the optoacoustic probe is placed in the interface. In certain embodiments, a force transducer is attached to a surface of the optoacoustic probe in order to measure the amount of force (tissue displacement) required in order to obtain the peak and optimal signal from a light source such as a pulsed laser diode (“PLD”). Optoacoustic measurements e.g., depth of vessel and oxygenation calculations will be continuously recorded after peak signal is obtained. All measurements are non-invasive.

Pulmonary artery catheterization and monitoring are standard of care for cardiac surgery. After obtaining written informed consent from subjects, an introducer sheath (such as for example, an 8.5 French Cordis sheath or the like) is placed into the left internal jugular in the OR under general anesthesia or sedation. The PA catheter (such as for example an Edwards LifeScience PA catheter or the like) is placed with the tip of the catheter located in the pulmonary artery and confirmed by PA occlusive waveform. This approach for comparing oxygenation measurements is shown in FIG. 10. The PA catheter has three ports that include the infusion port (super vena cava), proximal port (right atrium) and distal port (pulmonary artery). Referring to FIG. 10, showing the addition of a white Cordis introducer sheath that terminates with a port in the LIV (indicated by *). The PA catheter has three ports; the infusion port located in the superior vena cava (SVC), as indicated by 41, proximal port located in right atrium (RA) as indicated by 42 and distal port located in the PA as indicated by 43.

The introducer sheath (which is 15 cm in length in the case of a Cordis catheter), has one port. Based on the length and placement, this nearly guarantees that the tip of the introducer sheath catheter will be located in the innominate vein. Once the catheters are secured, ultrasound is used to confirm that the tip is located in the LIV. Ultrasound measurements may also determine the depth of the LIV from surface of skin. Determination of the distance, in mm, from the introducer sheath tip to genu of the left IJV may be made. In order to compare venous oxygenation from different sites, blood is sampled from the introducer in the LIV, SVC, and PA (representing mixed venous) and sent for hemoximetry (such as for example using an IL 682 Co-Oximeter, Instrument Laboratories, Bedford Mass.). In certain embodiments, venous blood samples are performed at distinct time points or periods for each subject. For example, specific time points may include: 1) baseline, which is defined after catheter placement but before surgery, 2) at end of surgery but prior to ICU transport, 3) one hour after ICU arrival, 4) post-operative day 1 in ICU before extubation, and 5) post-operative day 1 in ICU after extubation and immediately prior to removing the PA catheter. Data analysis is conducted to confirm that sampling the left innominate vein (LIV) correlates strongly with concurrent superior vena cava (SVC) oxygen saturation over perioperative course in a wide population sample. Data will be compared by linear regression with S_{LIV}O₂ (hemoximetry) plotted on the Y-axis versus in SSVCO₂ on the X-axis.

In one embodiment, in the same subjects outlined above, hemoximetry samples from the introducer port are compared to innominate vein saturation [S_{LIV}O₂] measured optoacoustically. The optoacoustic probe is placed in the suprasternal notch and directed towards the left innominate vein as previously described. Optoacoustic signal acquisition is done for 2-3 minutes to ensure adequate sampling time. For each optoacoustic measurement, the mean and standard deviation are performed. Each subject will undergo comparative measurements for optoacoustics and hemoximetry at the time points outlined previously. Blood from PA site will also be compared as this represents mixed venous blood.

S_{LIV}O₂ measured by optoacoustics is compared to determine strong correlation with simultaneous measurements of LIVO₂ saturation measured via hemoximetry. For example, data may be compared by linear regression, in which optoacoustic S_{LIV}O₂ are plotted on the Y-axis versus hemoximetry LIVO₂ saturation on the X-axis. In certain embodiments, measurements are compared using the Bland-Altman approach, in which the difference between S_{LIV}O₂ and _{LIV}O₂ saturation is compared to the average of the two measurements. This analysis of the agreement between the two measurements generates an estimate of the bias and precision between the measurements. A fairly wide distribution of values is expected due to different perioperative loading conditions and other situations e.g., paralysis, body temperature and bleeding (LIVO₂ saturation range from 45%-85% saturation is likely).

### Example 4—Optoacoustic Determination of S, v0₂ in Healthy Volunteers) to Simulate Hypovolemic Shock

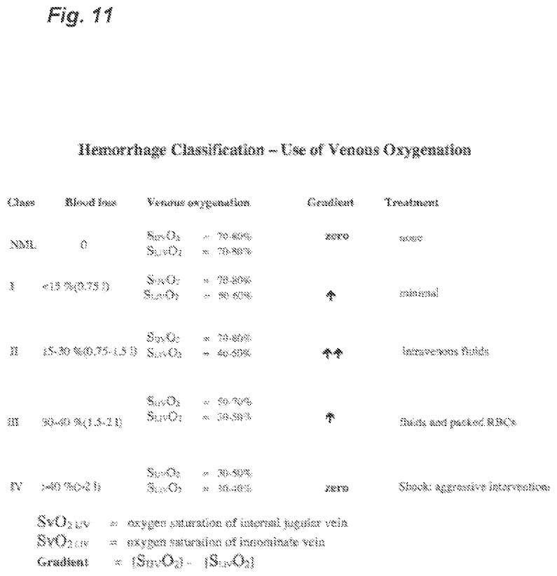

FIG. 11 depicts hemorrhage classifications based on determinations of venous oxygenation. Traditional estimates of blood loss rely on vital signs, which are often late findings. In the embodiment depicted in FIG. 11, venous oxygen saturation and gradient [difference between central perfusion e.g., IJV saturation and peripheral e.g., LIV saturation] are employed to approximate volume loss severity and physiologic compensation.

Use of Venous Oxygenation to Determine Hemorrhage Severity: It has long been recognized that hemorrhage causes an inadequate delivery of oxygen delivery to the tissues, due to lower Hgb and decreased cardiac output. Strong compensatory mechanisms are initiated at the onset of blood loss and hypovolemia. Activation of the autonomic nervous system, in particular sympathetic nervous system, results in blood flow centralization in order to secure perfusion of brain and heart, since these organs may not tolerate an interrupted supply of oxygen delivery. Conversely, sympathetic vasoconstriction leads to a reduction in perfusion of peripheral organs such as skin and muscle, which may adapt for longer periods of time with substantially lower blood flow, albeit at the expense of a lower tissue oxygen content. The amount of oxygen utilization may be estimated from the venous effluent oxygen content for each tissue. Specifically, the amount of oxygen in the regional venous system provides a direct gauge of perfusion to that organ.

For example, the amount of oxygen in the internal jugular vein is an index of brain perfusion, whereas the innominate vein oxygenation is an index of the upper thoracic cavity, which has significant muscle mass.

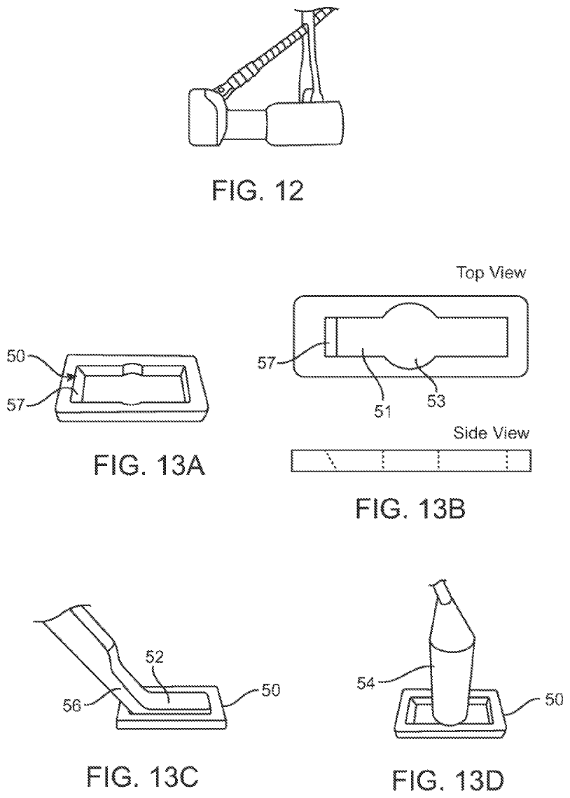

In certain embodiments, further clinical validation protocols are used to establish efficacy of a device and method for ultrasound guided optoacoustic monitoring of oxygen saturation to confirm that by measuring the venous oxygenation from a centralized source such as IJV and a source representing peripheral tissues such as LIV along with its differential gradient (centralized minus peripheral tissues), novel data is collected on hemorrhage severity of hemorrhage and its compensatory physiologic response (FIG. 12). Conceptually, due to vasoconstriction, peripheral venous oxygenation will precipitously decrease as hemorrhage severity is increased, whereas centralized venous oxygen is preserved until later stages of hemorrhage. The venous oxygenation gradient would therefore be increased during the compensatory phases. As hemorrhage severity continues, centralized flow becomes compromised. At this point, saturation in IJV declines and the gradient becomes pseudo-normalized.

Instrumentation, procedures, and measurements: Specifically, on the day of the human study, subject will be placed supine on a specialized mattress with their lower body sealed at the iliac crest inside the lower body negative pressure chamber. An 18-gauge peripheral i.v. catheter is placed in a hand or arm vein. A 20-gauge angiocatheter is inserted into the radial artery to measure arterial oxygenation SaO₂ and blood pressure, after an Allen's test to ensure radial and ulna collateral flow. Catheters are placed aseptically and secured in place with tape. After a sterile prep and drape and infiltration of local anesthesia, an oximetric pulmonary artery (“PA”) catheter (PreSep Edwards Life-Sciences Irving CA) is placed under ultrasound guidance in the left internal jugular vein via lateral border of the sternocleidomastoid muscle. Confirmation on pulmonary artery placement is assured via progression of right ventricular waveform, followed by a pulmonary waveform and lastly an occlusive pressure [PAOP] waveform when the balloon is inflated. After PAOP waveform is confirmed, the PA catheter is secured such as via suture. A thermodilution cardiac output (COTD) is performed to ensure confirmation that the curve has a right ventricular ejection pattern. All catheters are kept patent throughout the protocol with sterile saline solution in a pressurized bag.

In certain embodiments, throughout the entire protocol the variables are measured continuously including one or more of: invasive mean arterial blood pressure (MAP) via the arterial line, peripheral venous pressure (PVP), heart rate (HR) measured via an electrocardiogram (ECG; General Health Care), central venous pressure (CVP), pulmonary artery pressure (PAP), oximetric pulmonary artery saturation (SvO₂) and blood temperature. Ultrasound is used to mark the sites and define borders for target veins such as for example the IJV and LIV in order to more efficiently approach the target vessels for optoacoustic measurement. In brief, the optoacoustic probe is placed in the suprasternal notch to measure innominate vein S_{LIV}O₂ as described. An additional probe is placed on the lateral border of the stemocleidomastoid muscle to measure internal jugular vein saturation (SUVO₂). Echocardiography and hemoximetry is performed at time points described in FIG. 12.

Discharge: At D0 all lines will be removed and at D30, the subject is discharged.

Hemodynamic measurements: are recorded at time points such as T-60, T-30, TO, T5, T10, T15, T20, T25, T30, RO, R10 and R20—before discharge.

Arterial and venous Pressure: Continuous beat-by-beat arterial blood pressure is recorded invasively via a catheter in the radial artery. Mean arterial blood pressure (MAP) is calculated and recorded. Arterial blood pressure is digitally displayed and recorded at 1000 Hz via intra-arterial catheter transducer. Event times are noted and recorded on Powerlab software. The arterial catheter is also be used to measure arterial oxygenation (SaO₂) at specified time points. Similarly, a transducer is used to continuously measure CVP from the Pre-Sep catheter.

Electrocardiography (ECG heart rate): A normal clinical 3 lead ECG is placed on the subject's chest during the experimental procedure.

Temperature: Core blood temperature is obtained from the Pre-Sep catheter.

Pulse oximetry: Continuous pulse oximetry (SpO₂), perfusion indices (PVI and PI) and non-invasive Hgb—are continuously measured. The determinants provide which arterial blood saturation and perfusion.

Cardiac output (CO): is determined by thermodilution (injection of saline into proximal port and reading the thermodistribution from the distal port) from the PA catheter. Measurements are used to calculate systemic vascular resistance (SVR: dynes·sec·cm−5) as follows:

SVR=[MAP−CVP]/CO×80

Oxygen delivery (DO₂·) will be calculated from CO, Hgb and SO₂ as:

DO₂=CO×Hgb×1.3×SO₂

In certain embodiments, eligibility for the studies includes demonstration of good cardiac imaging in the two-chamber apical view. Where volunteers are young, free of cardiac disease, and have no regional wall motion abnormalities, it is anticipated that quantitatively reliable information from the two-chamber, apical view, will be obtained using the modified Simpson's rule to measure ventricular volume. End-diastolic (EDV) and end-systolic volume (ESV) measurements is obtained from a transducer and ultrasound system. A 3.5 MHz transducer and ultrasound system (Vivid 7 PRO BT04, GE Medical Systems, Milwaukee, Wis.) provides ultrasound location data in one embodiment. Left ventricular (LV) area and length is obtained from the parasternal LV long axis and used for volumetric calculations. In certain embodiments, the modified Simpson's rule is applied for calculating EDV, ESV, stroke volume (SV) and ejection fraction (EF %). Measurements are determined at all specified time points.

Co-oximetry for arterial and venous oxygenation: In certain embodiments, blood is sampled from arterial (SaO₂) and venous catheters (SvO₂) at time points including T-30, TO, T10, T20, T30, RO and R20 and measured using a co-oximeter. In certain embodiments, a volume, such as for example, 1 mL, of blood is removed from arterial and venous catheters, which are connected to the transducers.

Non-invasive optoacoustic determination of Venous Saturation: After mapping location using surface ultrasound, an optoacoustic probe is placed in the lateral border of the left suprastemal notch to measure the S_{LIV}O₂. A second optoacoustic probe is positioned on the left lower anterior triangle to measure SUVO₂. For each time point outlined (FIG. 12), signals generated over a 75 second window are averaged. The mean±SD for each measurement set is compared to venous hemoximetry samples. The S_{LIV}O₂ and S_{LIV}O₂ and gradient is also used to estimate hemorrhage severity and compensatory response.

Statistical considerations and data analysis: Statistical analysis is performed. Descriptive statistics are used such as for the analysis of the mean and standard error of the mean. A regression analysis is done for optoacoustic measurements versus hemoximetry.

### Example 5—Optoacoustically Measured Sₗᵢᵥ0₂ Correlates with Simultaneous Measurements Hemoximetry Derived SSVCO2

In certain embodiments, further clinical validation protocols are used to establish efficacy of a device and method for ultrasound guided optoacoustic monitoring of oxygen saturation. In one embodiment, an optoacoustic technique is applied to compare sets of data at various time points by linear regression (optoacoustic S_{LIV}O₂-Y-axis versus hemoximetry SSVCO₂ saturation-X-axis). A Bland-Altman approach may be performed. In human clinical validation protocols, a very good correlation is demonstrated clinically. Values of sensitivity, specificity and positive predictive value are obtained. While the adequate sample size for comparative studies is difficult to determine, especially based on the assumption that the two measurements are expected to have little difference between them. Therefore, requisite data points will be obtained to provide a broad range based on individual variability as well as baseline to pre-syncope as a model of real-world circumstances.

### Example 6—Simultaneous Measurement of the S_{LIV}O₂ and S_{LIV}O₂ to Determine the Venous Oxygenation Gradient

In certain embodiments, further clinical validation protocols are used to establish efficacy of a device and method for ultrasound guided optoacoustic monitoring of oxygen saturation to rapidly assess venous oxygenation changes that occur during a progressive simulated hemorrhage. Venous oxygen desaturation occurs when perfusion is reduced. This process takes minutes. While blood pressure is maintained due to compensatory increases in peripheral resistance, skeletal muscle mass (likely represented by S_{LIV}O₂) will continue to have low oxygen delivery and therefore oxygen debt increases leading to lower S_{LIV}O₂. Therefore, it is expected that S_{LIV}O₂ will likely continue to fall during the compensatory phase (venous oxygen gradient sub-hypothesis) even as it could be difficult to predict when decompensation phase occurs.

Provided herein is a novel, noninvasive, optoacoustic monitoring system that measures key indices of oxygenation that are altered with shock and TBI. Also provided are clinical validation protocols that establish efficacy of a device and method for ultrasound guided optoacoustic monitoring of oxygen saturation. In certain embodiments, human clinical trials are undertaken to evaluate the predictive value of S_{LIV}O₂ to diagnose shock and guide resuscitative therapy so that under and over-resuscitation do not occur. In certain embodiments, complementary determination of SIJvO₂ is also performed, which may provide: 1) new information on hemorrhage compensation 2) critical brain oxygenation data in subjects with TBI. In certain embodiments, the SO₂ data provided by the method and apparatus disclosed herein is used in conjunction with other modalities including: optimizing positive end-expiratory pressure (“PEEP”) [SpO₂ vs S_{LIV}O₂]; selecting vital fluid choices such as the need for blood transfusion vs other fluids; reducing oxygen consumption needs (considerations of the degree of fever and shivering vs a need for paralysis fluid and sedation and anesthesia); and duration and/or position of resuscitative endovascular balloon occlusion of an aorta (“REBOA”) to modulate proximal vs distal venous oxygenation.

### Example 7−Probe Operational Modes and Apparatus Examples

Mode 1: Sequential use of ultrasound guidance and optoacoustic measurement. In this sequential mode, first an ultrasound imaging (or Doppler measurements) is performed to localize a blood vessel of interest. Once the optimal location for monitoring of central venous oxygenation is identified, the optoacoustic probe is applied to provide oxygenation measurements of the identified blood vessels. The successive approach may be visual, i.e. visual identification of the target vessel with ultrasound image first, then optoacoustic measurements with an optoacoustic probe. FIG. 12 shows an optoacoustic probe that has been tested in CABG subjects.

Optoacoustic probes may be used in succession with various types of ultrasound probes. The ultrasound imaging probe i1 2L-RS (GE) was tested for vessel localization in conjunction with a GE Vivid system in the studies described in EXAMPLE 2. The GE ultrasound imaging probe i12L-RS has a wide frequency band of 5-13 MHz. Other U/S probes that have been tested successfully including the Doppler probe IPP3 having a frequency of 8 MHz and the Doppler probe VP4HS having a frequency of 4 MHz, but these particular U/S probes are given only as non-limiting examples.

A specially designed holder (subject interface) is preferably used for this purpose. The ultrasound probe is inserted in the holder and after the ultrasound procedure, the probe is removed from the holder and an optoacoustic probe is inserted in the holder to probe the blood vessel with high resolution and accuracy. The holder structure allows for sequential use of the ultrasound probe and optoacoustic probe at the same tissue site. The axis of the optoacoustic probe may coincide with that of the ultrasound probe. Using this mode, ultrasound-guided optoacoustic monitoring of blood oxygenation was performed in the innominate and other veins. This mode and data generated thereby was demonstrated in EXAMPLE 2 herein.

In certain embodiments, disposable adapters or subject interfaces are provided for successive (or iterative) use of ultrasound and optoacoustic probes. FIGS. 13A-13C show an example of use of such an adapter. As shown in FIG. 13A, the geometry of the adapter 50 allows for holding and inserting ultrasound and optoacoustic probes successively in the same location on the subject. First as depicted in FIG. 13C, an ultrasound probe (52) nestled into adapter (50) is used to find the blood vessel of interest. Once the blood vessel is found and optimal location of the optoacoustic probe is identified, adapter (50) is attached to skin of the subject at the optimal location using a medical adhesive or tape. Then, as shown in FIG. 13D, the ultrasound probe is removed from the adapter and the optoacoustic probe (54) is inserted in the holder. As depicted in FIG. 13B, holder (50) includes a space (51) dimensioned to approximate and securely hold ultrasound probe (52) depending on the geometry of the probe. Also as depicted in FIG. 13B, holder (50) also includes a space (53) dimensioned to approximate and securely hold an optoacoustic probe depending on the geometry of the probe. The exemplified ultrasound probe (52) is GE i1 2L-RS intraoperative linear probe (General Electric, Milwaukie Wis.) and includes a sloping “wand” like handle (56). Thus, as depicted in FIGS. 13A and 13B, holder (50) includes a sloping rest (57) that further customizes the holder to the geometry of the probe to be used. Because the axis of the optoacoustic probe is aligned with axis of the ultrasound probe using the holder, the optoacoustic detection of the blood vessel signals is optimal when the optoacoustic probe is inserted in the adapter. After the procedure, the adapter may be disposed of

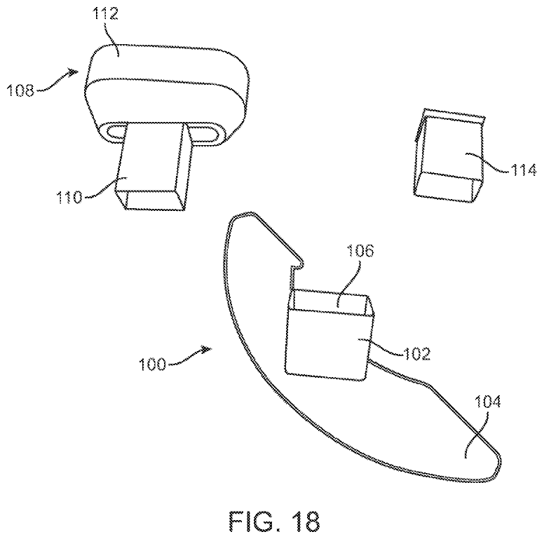

FIGS. 18-20 depict non-limiting examples of an apparatus (100) comprising an adapter (102) configured for successive use of an ultrasound probe and optoacoustic probe. In some embodiments, the apparatus (100) comprises a base (104) coupled to the adapter (102) and that is configured to be positioned against a portion of a subject body (“anatomical portion”), such as the chest, neck, torso, arm, leg, etc. As used herein, the term “portion of a subject body”, “anatomical portion”, and “anatomical portion of a subject” are used interchangeably. In some embodiments, the adapter (102) comprises a housing having a proximal opening (106) located at a proximal end of the housing, and a distal opening (107) located at a distal end of the housing. In some embodiments, the adapter extends through the base. In some embodiments, the distal end of the adapter housing is planar with the base.

In some embodiments, the base (104) is rigid and inflexible. In some embodiments, the base is flexible. In some embodiments, the base is rigid but configured to be flexed or deformed upon being subject to a minimum level of stress (e.g., the base is configured to be bent). In some embodiments, the base comprises an adhesive on the side of the base opposite the side where the proximal end of the adapter is located. In some embodiments, the adhesive is configured to attach the base to the anatomical portion of the subject. In some embodiments the adhesive comprises a medical grade adhesive. In some embodiments, the adhesive comprises a two-sided medical grade adhesive. In some embodiments, the base is attached to the anatomical portion via other means known in the art.

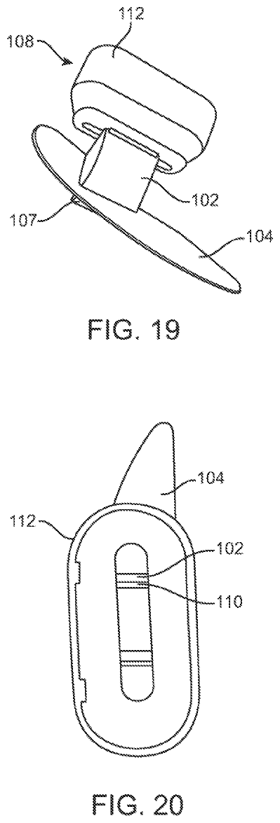

In some embodiments, the adapter (102) is configured to receive at least a portion of an ultrasound probe through the proximal opening (106). The ultrasound probe may be any ultrasound probe as described herein or known in the art. In some embodiments, the adapter (102) is also configured to receive at least a portion of an optoacoustic probe through the proximal opening (106). The optoacoustic probe may be any optoacoustic probe as described herein or known in the art. In some embodiments, the adapter is configured to hold the ultrasound probe in a position when the base is placed against anatomical portion of a subject. In some embodiments, the adapter is also configured to hold the optoacoustic probe in a position when the base is placed against an anatomical portion of a subject. In some embodiments, at least a distal portion of the ultrasound probe comprises a similar form factor (e.g., shape, size, dimension) to at least a distal portion of the optoacoustic probe, thereby enabling the same adapter (102) to be configured for successive use of said ultrasound probe and said optoacoustic probe. In some embodiments, the ultrasound probe is inserted into an ultrasound probe extension (UPE) (108). In some embodiments, the UPE comprises a proximal portion (112) configured to receive the ultrasound probe, and a distal portion (110) configured to be inserted into the adapter (102) via the proximal opening (106). In some embodiments, the distal portion (110) of the UPE is detachably coupled with the proximal portion (112). FIG. 18 depicts the UPE (108) and adapter (102) separated, while FIGS. 19-20 depicts the distal portion (110) of the UPE (108) inserted into the adapter (102). Specifically, FIG. 19 depicts a perspective view of the coupling between the UPE (108) and adapter (102), while FIG. 20 depicts a top view of the UPE (108) and adapter (102), wherein the distal portion (110) of the UPE is shown to be inserted within the adapter. FIG. 20 further depicts a top view of the proximal portion (112) of the UPE configured for receiving the ultrasound probe.

As depicted in FIG. 18, in some embodiments, the optoacoustic probe is inserted within an optoacoustic probe extension (OPE) (114), which is configured to be inserted into the adapter (102) through the proximal opening (106). In some embodiments, the OPE (114) is the same component as the distal portion (110) of the UPE (108), which is used for both the ultrasound probe and optoacoustic probe.

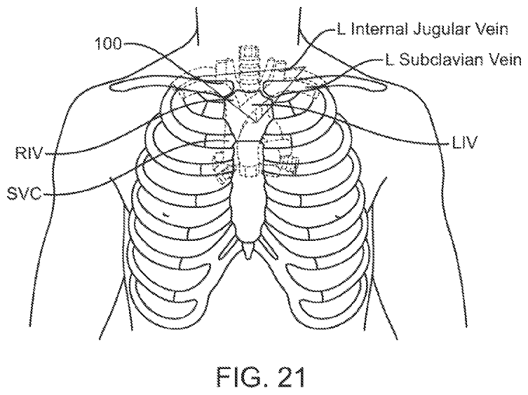

As depicted in FIG. 20, the distal opening (107) (see FIG. 19) of the adapter is visible despite the insertion of the UPE into the adapter. In some embodiments, the field of view of the ultrasonic probe is aligned with the distal opening (107) so as to locate a targeted blood vessel, as described herein. Accordingly, the apparatus (100) may be moved and placed at various locations until the ultrasound probe locates a targeted blood vessel, at which point the base may be attached to the respective anatomical portion to secure the adapter positioning, and wherein the ultrasound probe is removed, and the optoacoustic probe is then inserted into the adapter and aligned with the targeted blood vessel. In some embodiments, the ultrasound probe locates the target blood vessel by obtaining an ultrasound image of an anatomical portion and verifying the presence of the target blood vessel in the image. FIG. 21 depicts an exemplary placement of the apparatus at the sternal notch, wherein the adapter is positioned to align the distal opening (107) with the left innominate vein (the target blood vessel in this case). In some embodiments, the target blood vessel is a left innominate vein, a right innominate vein, a superior vena cava, an aorta, a right internal jugular vein, a left internal jugular vein, a left subclavian vein, a right subclavian vein, or a combination thereof. In some embodiments, the optoacoustic probe is positioned to measure blood oxygenation in a target tissue in the body.

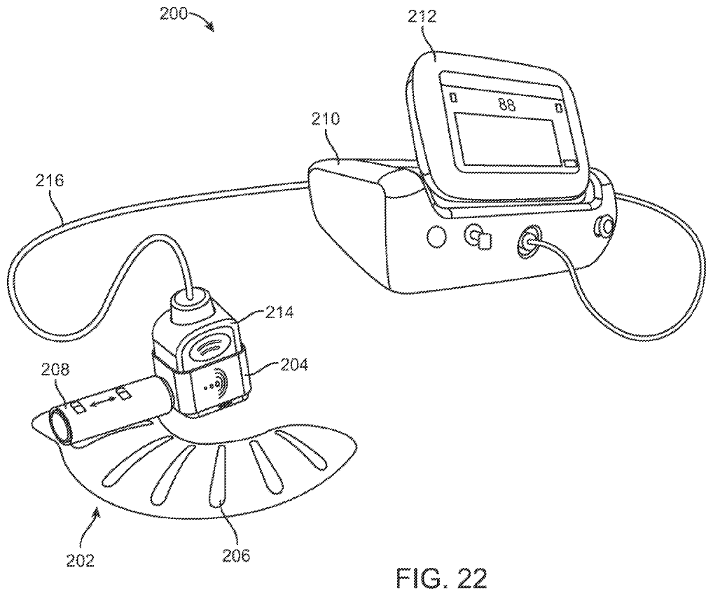

FIGS. 22-25 depict a non-limiting example of a system (200) and components for ultrasound guided optoacoustic measurement of blood oxygenation in a blood vessel. As depicted in FIG. 22, in some embodiments, the system (200) comprises an ultrasound probe (not shown) and an optoacoustic probe (e.g., 214), an apparatus (202) for positioning and/or aligning the ultrasound probe and/or optoacoustic probe to a target blood vessel at a subject site, and a system controller (210) operatively coupled with the ultrasound probe and/or optoacoustic probe. In some embodiments, the system controller (210) comprises an interface for outputting images and/or measurement data obtained via the ultrasound probe and/or optoacoustic probe. In some embodiments, the said system controller interface comprises a display (212) for outputting the obtained imaging and measured data. In some embodiments, the system controller (210) is configured to receive input from a user via the interface. In some embodiments the system controller (210) is in electrical communication with the ultrasound probe and optoacoustic probe (e.g., 214), either simultaneously or one at a time. In some embodiments, the system controller (210) provides power to the ultrasound probe and optoacoustic probe, either simultaneously or one at a time. In some embodiments, the system controller provides instructions (e.g., on/off, activation/deactivation) to the ultrasound probe and/or optoacoustic probe.

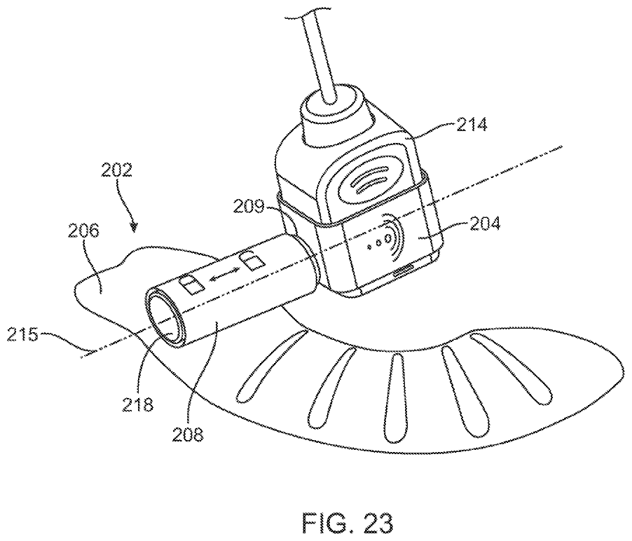

FIG. 23 depicts a non-limiting example of an apparatus (202) with an optoacoustic probe (214) coupled thereto. The optoacoustic probe may be any optoacoustic probe described herein. In some embodiments, as depicted in FIG. 23, the optoacoustic probe (214) is operatively coupled to the system controller (210) as well. In some embodiments, the apparatus comprises a base (206), an anchor (208) coupled to the base (206) and defining a cavity within, a shaft (209) that at least partially extends into anchor cavity, and an adapter (204) that is coupled to the shaft. In some embodiments, the base is flexible. In some embodiments, the flexibility of the base enables its placement over an anatomical structure of a subject, wherein the base is configured to conform to the anatomical structure. The base may be placed over any anatomical portion of a subject, such as a chest, neck, torso, back, head, arm, leg, etc. In some embodiments, the anatomical portion includes a chest of a subject, such as the upper chest, or an anatomical portion just below the neck. In some embodiments, the base is rigid. In some embodiments, the shape of the base is configured to be manipulated upon being placed under a minimum level of stress. For example, in some embodiments, the base may be rigid but bendable. In some embodiments, the base is curved as depicted in FIG. 23. In some embodiments, the base is any shape, size and/or configuration. In some embodiments, the base comprises a shape of a square, rectangle, circle, or any other polygonal shape. In some embodiments, the base comprises an adhesive to enable attachment to an anatomical portion of a subject. In some embodiments, the adhesive is a medical grade adhesive. In some the adhesive is a two-sided adhesive.

In some embodiments, the anchor is detachably coupled to the base. In some embodiments, the anchor is rigidly coupled to the base. In some embodiments, the anchor extends from a side of the base opposite to the side where the base couples to anatomical structure of the subject. In some embodiments the anchor is of any shape, such as cylindrical, spherical, cuboidal, rectangular, etc. In some embodiments, the anchor defines a cavity within. In some embodiment, the cavity extends from one end of the anchor and at least partially through the anchor. In some embodiments, the cavity extends between both ends of the housing.

In some embodiments, the shaft (209) extends partially through the anchor cavity. In some embodiments, the shaft extends from one end of the anchor and through the other end of the anchor. In some embodiments, the shaft is rotatable about a longitudinal axis (215).

In some embodiments the adapter comprises a housing configured to receive an ultrasound probe and an optoacoustic probe, either simultaneously or one at a time. In some embodiments, as described herein, the ultrasound probe and/or optoacoustic probe is provided in an extension that is configured to receive said ultrasound and/or optoacoustic probed, and wherein the respective extension is configured to be inserted in the adapter housing (204). In some embodiments, the adapter housing comprises a proximal opening located at a proximal end of the adapter housing, and a distal opening located at a distal end of the adapter housing, defining a channel between the proximal and distal openings. In some embodiments, a dimension (e.g., diameter, cross-section) of the proximal opening is the same as a dimension of the distal opening of the adapter housing. In some embodiments, a maximum dimension of the proximal opening is larger than a maximum dimension of the distal opening. In some embodiments, the maximum dimension comprises a diameter, cross-section, cross-sectional area, etc. In some embodiments, the ultrasound probe (or corresponding extension), and/or the optoacoustic probe (or corresponding extension) is inserted within the adapter housing via the proximal opening. In some embodiments, the channel within the adapter housing tapers inwards from the proximal opening to the distal opening, such that the ultrasound probe (or corresponding extension) and/or optoacoustic probe (or corresponding extension) are prevented from entirely sliding out the distal end (i.e. providing a friction fit).

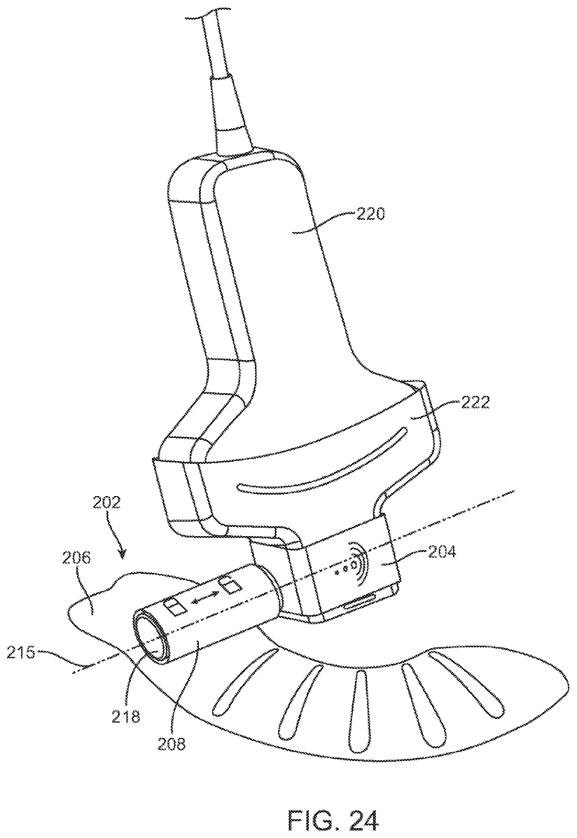

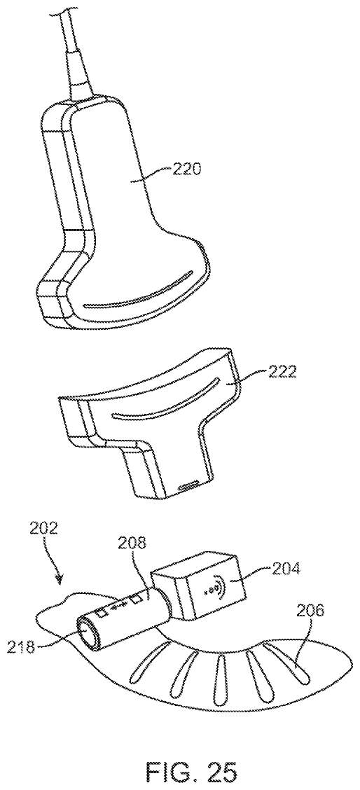

In some embodiments, the ultrasound probe is configured to be received within the adapter housing (204). The ultrasound probe may be any ultrasound probe described herein. In some embodiments, as depicted in FIGS. 24-25 and as described herein, the ultrasound probe (220) is configured to be coupled to an extension (222) that is received within the adapter housing (204), such that the ultrasound probe may be positioned by the adapter housing. In some embodiments, the extension comprises a proximal end configured to receive and mate with a distal end of the ultrasound probe, and the extension further comprises a distal end configured to be received and mate with the adapter housing (204). In some embodiments, at least the distal portion of the extension for the ultrasound probe and the distal portion of the optoacoustic probe comprise the same or similar form factor (e.g., similar shape, size, dimensions, and/or depth) such that the adapter (204) is configured to hold the ultrasound probe (e.g., via the extension) and the optoacoustic probe one at a time. In some embodiments, at least the distal portion of the ultrasound probe and the distal portion of the optoacoustic probe comprise the same or similar form factor (e.g., similar shape, size, dimensions, and/or depth) such that the adapter (204) is configured to hold the ultrasound probe (e.g., via the extension) and the optoacoustic probe one at a time.

In some embodiments, the adapter housing (204) is coupled to the anchor (208) via the shaft (209). In some embodiments, the shaft (209) is detachably coupled to the adapter (204). In some embodiments, the shaft is part of the adapter (e.g., an extension therefrom). In some embodiments, the adapter (204) is rotatable about the longitudinal axis (215) via the shaft (209). In some embodiments, the adapter (204) rotates in concert with the shaft (209) and relative to the anchor (208) and base (206). In some embodiments, the adapter is configured to rotate 360 degrees. In some embodiments, rotating the adapter housing (204) enables the ultrasound probe and/or optoacoustic probe to be aligned (e.g., field of view alignment) with an anatomical portion of a subject at various angles, when the base is placed against the anatomical portion. For example, depending on where the base is disposed against the subject, the ultrasound probe may be angled, via rotation of the adapter (204), thereby adjusting an angle of alignment (e.g., of the field of view) between the ultrasound probe and anatomical portion of the subject, which corresponds to an angle of alignment between the adapter (204) and anatomical portion of the subject. As such, an optimal angle of alignment between the ultrasound probe and anatomical portion of the subject may be identified that correlates with a location for a target blood vessel (as described herein, e.g., left innominate vein), as determined via ultrasound imaging obtained from the ultrasound probe. In some embodiments, the angle of alignment between the ultrasound probe and anatomical portion may be adjusted from 0 degrees to 360 degrees. In some embodiments, the angle of alignment between the ultrasound probe and anatomical portion is restricted to the rotational movement of the adapter. In some embodiments, the adapter is restricted to rotate from about 90 degrees to about 270 degrees. In some embodiments, the adapter is restricted to rotate about 150 degrees. In some embodiments, the adapter is restricted to rotate about 75 degrees in either direction (e.g., clockwise or counterclockwise) relative to the anatomical portion. In some embodiments, a locking mechanism, as described herein, provides the restriction of the rotational movement of the adapter.

In some embodiments, the rotational position of the adapter (204) relative to the anchor (208) and base (206), may be secured using a locking mechanism, thereby further prevent rotation of the adapter (204). In some embodiments, the anchor comprises a push button that activates the locking mechanism. In some embodiments, the locking mechanism comprises a clamp that clamps the shaft (209) against the anchor (208), thereby preventing further rotation of the shaft (209). As such, by locking the rotational position of the adapter (204) (relative to the anchor and base), the adapter may secure a desired angle of alignment between an ultrasound probe and/or optoacoustic probe, such that a user or administrator need not hold the adapter rotational position in place to maintain alignment when measuring blood oxygenation levels intervention. Moreover, this helps improve the effectiveness of measuring the blood oxygenation level by inadvertent misalignment by a user or administrator when using the apparatus. In some embodiments, the push-button is spring loaded. In some embodiments, the push-button the spring loaded configuration enables one-handed operation to lock and unlock the rotational position of the adapter.

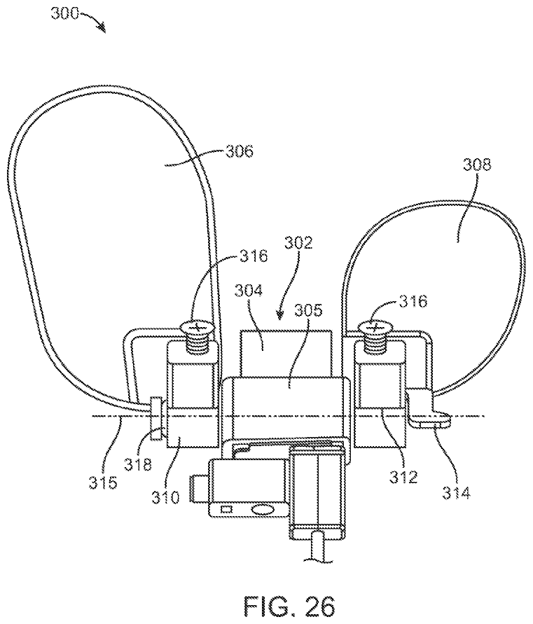

FIG. 26 depicts another non-limiting example of an apparatus (300) for ultrasound guided optoacoustic measurement of blood oxygenation in a blood vessel. As described herein, an anatomical portion includes a chest of a subject, such as the upper chest, or an anatomical portion just below the neck.

In some embodiments, as depicted in FIG. 26, the apparatus (300) comprises a left chest wing (306), a right chest wing (308), a left anchor (310), a right anchor (312), a shaft (318), and an adapter (302) rotationally coupled to the left and right chest wings via the shaft and left and right anchors, wherein the adapter is configured to receive an ultrasound probe and optoacoustic probe, either simultaneously or one at a time (FIG. 26 provides an exemplary depiction of the adapter receiving an optoacoustic probe).

In some embodiments, the left chest wing (306) and/or right chest wing (308) are configured for placement against the corresponding chest of a subject. In some embodiments the left and/or right chest wings are configured to be flexible, rigid, or deformed upon being subject to a minimum level of stress. In some embodiments, the left and/or right chest wing comprises a two-sided medical-grade adhesive to secure placement against the chest of a subject. In some embodiments, the left anchor (310) is coupled to the left chest wing (306), and the right anchor (312) is coupled to the right chest wing (308), wherein the left and right chest wings are coupled together via the shaft (318) and adapter (302). In some embodiments, the shaft (318) is configured to be rotated about a longitudinal axis (315) and relative to the left and right anchors (310, 312). In some embodiments, the shaft (318) extends through and out of the left and right anchors. In some embodiments, the shaft (318) comprises a left portion extending from the adapter (302) and through the left anchor (310), and a right shaft portion, extending from the adapter (302) and through the right anchor (312). In some embodiments, the left and right shaft portions are detachably coupled to the adapter (302). In some embodiments, the left and right shaft portions are a part of the adapter (302) (e.g., an extension therefrom). In some embodiments, at least one end of the shaft comprises a thumb screw (314) to facilitate rotation of the shaft (318) by a user. In some embodiments, at least one of the left and right anchors comprises a screw (316) extending therethrough and configured to clamp the shaft (318) against the respective anchor (310, 312), thereby preventing further rotation of the shaft (318) and adapter (302).

In some embodiments, the adapter (302) is configured to rotate about the longitudinal axis (315) via the shaft (e.g., left and right shaft portions). In some embodiment, the adapter (302) moves in concert with the rotation of the shaft (318). In some embodiments, the adapter is configured to rotate 360 degrees. In some embodiments, the adapter is restricted to rotate from about 90 degrees to about 270 degrees. In some embodiments, the adapter is restricted to rotate about 150 degrees. In some embodiments, the adapter is restricted to rotate about 75 degrees in either direction (e.g., clockwise or counterclockwise) relative to the anatomical portion. In some embodiments, a locking mechanism, as described herein, provides the restriction of the rotational movement of the adapter.

In some embodiments, the adapter (302) comprises a housing, wherein the housing comprises a proximal portion (305) and a distal portion (304). In some embodiments, the proximal portion (305) has a proximal opening, through which the adapter (302) is configured to receive the ultrasound probe and optoacoustic probe, either simultaneously or one at a time. In some embodiments, the distal portion (304) comprises a distal opening. In some embodiments, an ultrasound probe, or ultrasound probe extension (UPE), as described herein, is configured to be received by the adapter through the proximal opening. In some embodiments, the ultrasound probe is configured to be aligned (e.g., field of view alignment) with an anatomical portion of a subject via the adapter (302), wherein an angle of alignment between the ultrasound probe and the anatomical portion may be adjusted via rotation of the adapter (302). In some embodiments, the angle of alignment between the ultrasound probe and anatomical portion may be adjusted from 0 degrees to 360 degrees. As described herein, in some embodiments, the angle of alignment between the ultrasound probe and anatomical portion is restricted to the rotational movement of the adapter. As described herein, the screw(s) (316) enables a rotational position of the adapter (302) to be locked, so as to prevent the adapter (302) from further rotation, and thereby enables a desired angle of alignment between the ultrasound probe and anatomical portion to be secured.

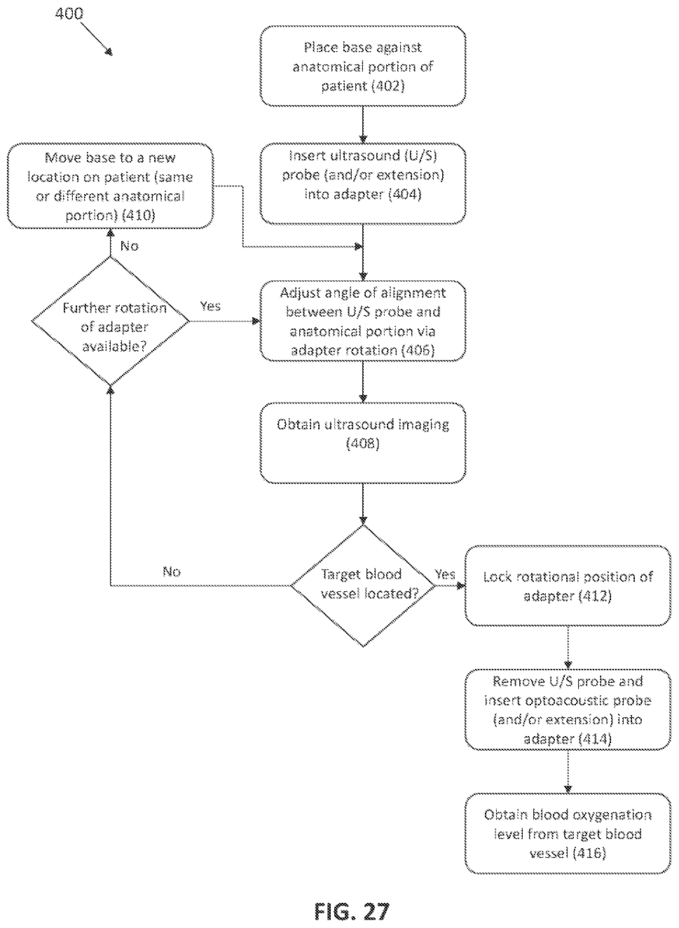

FIG. 27 depicts a flow chart for a non-limiting example of a method (400) for ultrasound guided optoacoustic measurement of blood oxygenation in a blood vessel. The method is applicable for any device described herein capable of articulating the angle of an ultrasound probe relative to a base, such as at least the apparatus depicted herein in FIGS. 22-26. As described herein, the target blood vessel may include a left innominate vein, a right innominate vein, a superior vena cava, an aorta, a right internal jugular vein, a left internal jugular vein, a left subclavian vein, a right subclavian vein, or a combination thereof.

With reference to FIG. 27, a base of an apparatus is first placed (step 402) against an anatomical portion of a subject. In some embodiments, the anatomical portion is the chest of a subject, such as the upper chest, a neck region, torso, back, head, arm, leg, etc. Once the base has been placed against the anatomical portion, an ultrasound probe is then positioned (step 404) by an adapter coupled to the base, wherein the ultrasound probe is positioned relative to the anatomical portion. The ultrasound probe may be any ultrasound probe as described herein. In some embodiments, the ultrasound probe is positioned by being inserted into an adapter housing. In some embodiments, the ultrasound probe is positioned by being inserted into an ultrasound probe extension (UPE), wherein the UPE is configured to be inserted into the adapter housing. Once the ultrasound probe has been positioned, the ultrasound probe may be aligned with the anatomical portion at a desired angle of alignment. In some embodiments, the desired angle of alignment will correspond to aligning the ultrasound probe with a location of the anatomical portion that aligns the ultrasound probe with a location of a target blood vessel. In some embodiments, the target blood vessel is a left innominate vein, a right innominate vein, a superior vena cava, an aorta, a right internal jugular vein, a left internal jugular vein, a left subclavian vein, a right subclavian vein, or a combination thereof. In some embodiments, the angle of alignment between the ultrasound probe and anatomical portion is adjusted (step 406) by rotating the adapter, so as to align the ultrasound probe with an estimated location of the target blood vessel.

Once the ultrasound probe has been aligned at the desired angle, the ultrasound probe is activated to obtain an ultrasound image (step 408) of the anatomical portion. In some embodiments, a system controller, as described herein (e.g., FIG. 22) is used to activate the ultrasound probe and display the image of the anatomical portion. If the obtained ultrasound image does not depict the target blood vessel, then the angle of alignment between the ultrasound probe and anatomical portion may be adjusted, by rotating the adapter, so as to identify a different angle of alignment to locate the target blood vessel. In some embodiments, the obtained ultrasound image depicts the target blood vessel but may correspond to an undesired alignment for blood oxygenation measurement by an optoacoustic probe (positioned with the same angle of alignment as the ultrasound probe). Accordingly, the adapter may be rotated so as to provide for a better angle of alignment between the optoacoustic probe and anatomical portion to provide a better alignment between the optoacoustic probe and target blood vessel. If the angle of alignment has already been adjusted a plurality of times, such that there are no other rotational positions of the adapter available to better align the ultrasound probe and target blood vessel, the apparatus may be moved (step 410) to another location on the subject, thereby re-starting the iteration for identifying an optimum angle of alignment between the ultrasound probe and anatomical portion that aligns with the target blood vessel. In some embodiments, the apparatus is moved to another location of the anatomical portion (e.g., shifting laterally on a subject's chest). In some embodiments, the apparatus is moved to another anatomical portion (e.g., moving from the chest to a neck portion). In some embodiments, the apparatus is moved so as to be placed over two or more anatomical portions simultaneously.

If the obtained ultrasound image depicts the target blood vessel, the rotational position of the adapter is subsequently locked (step 412) to prevent further rotation of the adapter. This enables the angle of alignment between the ultrasound probe and anatomical portion to be secured. In some embodiments, the rotational position of the adapter is locked via a locking mechanism described herein, such as in FIGS. 23-25, or the screws in FIG. 26. In some embodiments, the base is also secured to the subject, thereby reducing the risk of the adapter being displaced and compromising the identified angle of alignment between the ultrasound probe and anatomical feature. In some embodiments, the base is secured to the subject using an adhesive, such as a medical grade adhesive, a two-sided adhesive. Once the rotational position of the adapter has been secured, and optionally the base has been secured to the subject, the ultrasound probe may be removed from the adapter (or the UPE is removed), and an optoacoustic probe may be positioned (step 414) relative to the anatomical portion by the adapter. In some embodiments, the optoacoustic probe is positioned by being inserted into an adapter housing. In some embodiments, the optoacoustic probe is positioned by being inserted into an optoacoustic probe extension (OPE), wherein the OPE is inserted into the adapter housing. Since the rotational position of the adapter has been locked, the optoacoustic probe is aligned with the location on the anatomical portion that aligns the optoacoustic probe with the target blood vessel. Accordingly, the optoacoustic probe may be operated to measure the oxygenation (step 416) of the blood within the target blood vessel. In some embodiments, a system controller, as described herein (e.g., FIG. 22) is used to operate the optoacoustic probe and display the blood oxygenation level. In some embodiments, by locking the rotational position of the adapter, and optionally attaching the base to the subject, the measurement of blood oxygenation using the apparatus may be performed without the need for a user or administrator to hold the optoacoustic probe to maintain alignment with a target blood vessel.

In some embodiments, for the method described herein and in FIG. 27, the base is placed over a first anatomical portion of a subject, and the ultrasound probe is aligned with a second anatomical portion of the subject. In some embodiments, the first anatomical portion is a neck portion, and the second anatomical portion is a chest portion. In some embodiments, the first anatomical portion is a chest portion, and the second anatomical portion is a neck portion. In some embodiments, the angle of alignment refers to the alignment between the ultrasound probe (and/or optoacoustic probe) and second anatomical portion.

Although the above steps show method 400 for measuring one or more parameters from a subject or subject in accordance with many embodiments herein, a person of ordinary skill in the art will recognize many variations based on the teaching described herein. The steps may be completed in a different order. Steps may be added or deleted. Some of the steps may comprise sub-steps. Many of the steps may be repeated as often as beneficial to the measurement.

One or more of the steps of the method 400 may be performed with circuitry as described herein, for example, one or more of the processor or logic circuitry such as the programmable array logic for a field programmable gate array. The circuitry may be programmed to provide and/or facilitate one or more steps of the method 400, and the program may comprise program instructions stored on a computer readable memory or programmed steps of the logic circuitry such as the programmable array logic or the field programmable gate array, for example.

Mode 2: Dual mount ultrasound guidance and optoacoustic measurement apparatus. In the dual mount mode, both ultrasound probe and the optoacoustic probe are mounted together in a holder and once the blood vessel of interest is identified and localized with the included U/S probe, optoacoustic measurements are performed with the optoacoustic probe. Ultrasound imaging and optoacoustic measurements may also be performed simultaneously and continuously. The axis of the optoacoustic probe may be parallel to the axis of the ultrasound probe. Alternatively, the axis of the optoacoustic probe may be adjusted at some angle with respect to the axis of the ultrasound probe to provide accurate probing from a specific depth in tissue, in particular, from the depth of the blood vessel of interest.

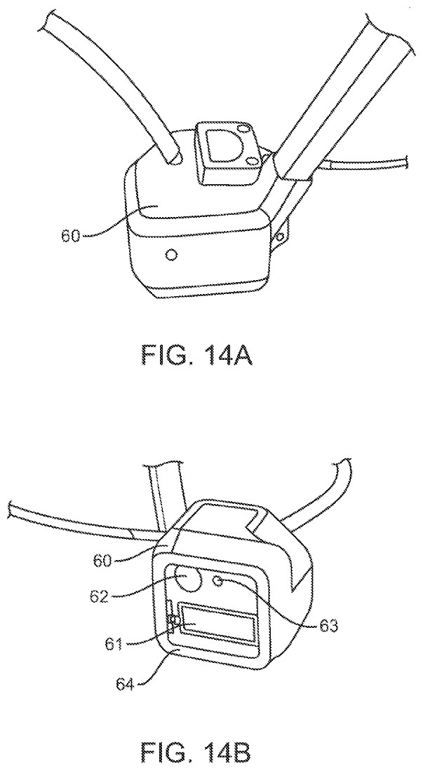

FIGS. 14A-14B depict combined ultrasound imaging and optoacoustic monitoring probes. An ultrasound imaging probe, specifically depicted is a Vivid e i12L-RS (GE) U/S probe, is combined in one casing (60) with a miniature optoacoustic probe. The elements of the dual apparatus are seen FIG. 14B, which shows the ultrasound probe face (61) and an optoacoustic probe with a sensitive element, larger circle (62), and optical fiber (63) for light delivery (the smaller dark circle). The bottom part (64) of the combined probe has an indentation which may be filled with a molded gel pad for acoustic matching to tissues.

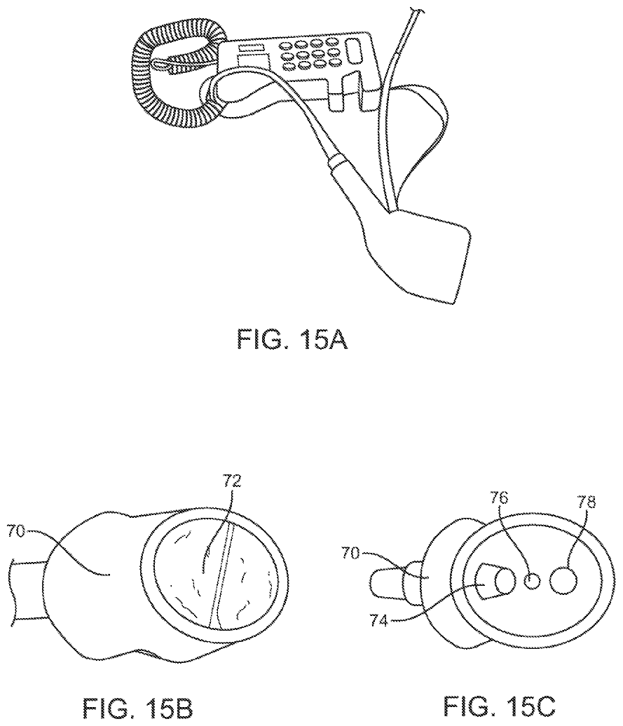

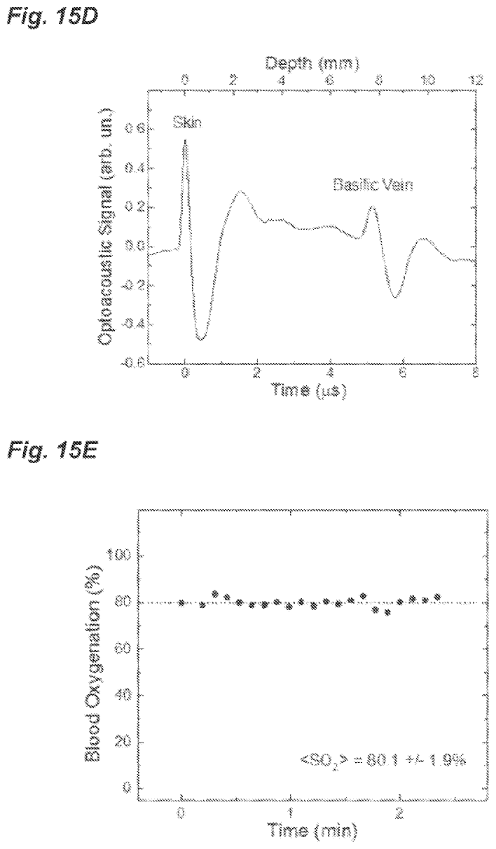

FIGS. 15A-15C depict combined ultrasound imaging and optoacoustic monitoring probes wherein a Doppler ultrasound system is adapted combination with for optoacoustic monitoring. In the depicted example of FIG. 15A, a combined instrument prototype is shown that has been constructed from a handheld Doppler ultrasound system (Model MD2 VP4HS (4 MHz probe, Huntleigh Technology Plc.) combined with an optoacoustic system by adding a light source and an optoacoustic transducer. As shown in FIG. 15B, the bottom part of the combined probe housing (70) may be covered with a molded gel pad (72). The casing of the combined probe allows for use of a bigger Doppler probe such as one like the VP4HS depicted (74). Alternatively, a pencil-like probe IPP3 (not shown) may utilized and securely mounted in the combined probe FIG. 15C. The optoacoustic transducer is inserted into hole (78). A pulsed laser light source will also be mounted in the housing for optoacoustic stimulation through hole (76). Using this prototype probe, optoacoustic signals and blood oxygenation in basilic vein were measured as shown in FIGS. 15D-15E. FIG. 15D shows an optoacoustic signal recorded from the basilic vein after it was detected with the Doppler probe. Continuous optoacoustic monitoring of the basilic vein oxygenation is presented in FIG. 15E. The average and standard deviation of the blood oxygenation were 80.1% and 1.9%.

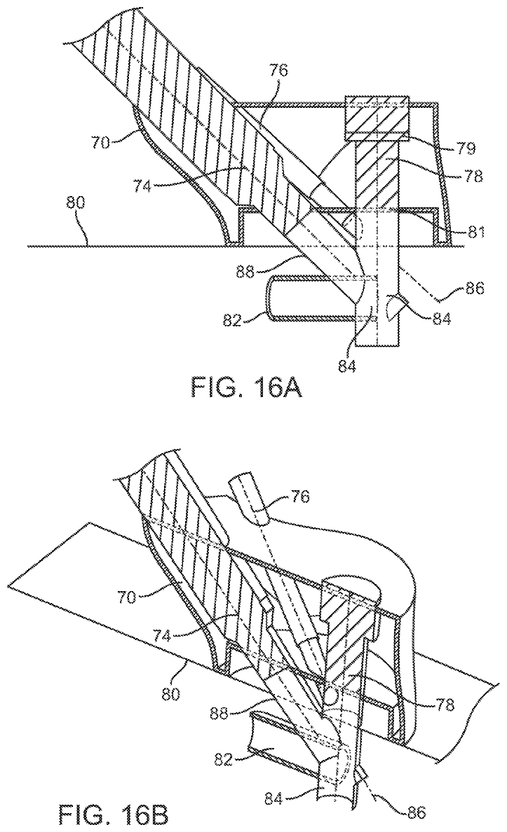

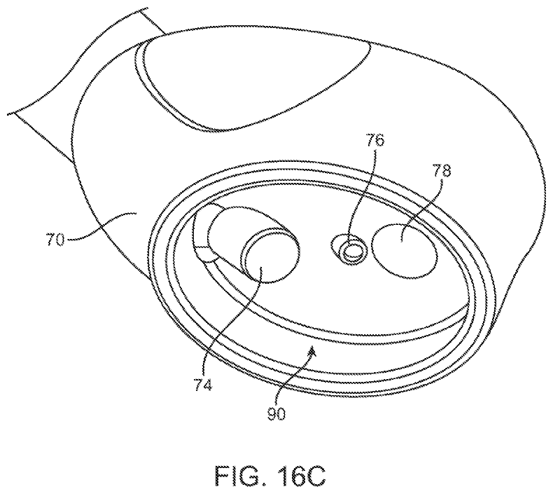

FIG. 16A-16B show side and oblique views respectively of two embodiments of dual mount ultrasound guidance and optoacoustic measurement apparatus. In both cases, holder (70) securely mounts ultrasound probe (74), light source (76) and optoacoustic probe (78). The optoacoustic probe (78) may include a printed circuit board (79) and other electronics. The face of the optoacoustic transducer is protected by a film (81) such as for example a polyguard film of 5-15 mil. In some embodiments the film is 10 mil. The skin of the subject is figuratively shown as (80) and the paths of investigation of the vein (82) by ultrasound field (88), optoacoustic stimulating light path (86) and optoacoustic investigation field (84). In FIG. 16A, the probes are positioned in the same plane and aligned at a specific angle to one another to provide optoacoustic probing from a specific depth and accurate measurement of blood oxygenation from this depth.

FIG. 16B depicts another embodiment of a dual ultrasound (or Doppler) probe and an optoacoustic probe. The probes are positioned in different planes and aligned at a specific angle to provide optoacoustic probing from a specific depth and accurate measurement of blood oxygenation from this depth.

FIG. 16C shows an oblique bottom view of an embodiment of a dual mount ultrasound guidance and optoacoustic measurement apparatus showing holder (70) securely mounting ultrasound probe (74), light source (76) and optoacoustic transducer (78). In the depicted example, holder (70) includes a hollow interior space (90), within which the internal components of the probe reside. In certain embodiments the hollow interior space is designed to be filled with an acoustic gel that contacts both the ultrasound and optoacoustic sensors.

An acoustic backing material may be positioned in the holder behind the optoacoustic transducer. It provides backing for the sensor (for wideband detection of pressure waves) and absorbs the vibrations that travel through the sensor to prevent undesired ringing in the signal and separate part of the signal from ringing noise. In some embodiments, the attenuator comprises a mass of a plastic material such as an epoxy material.

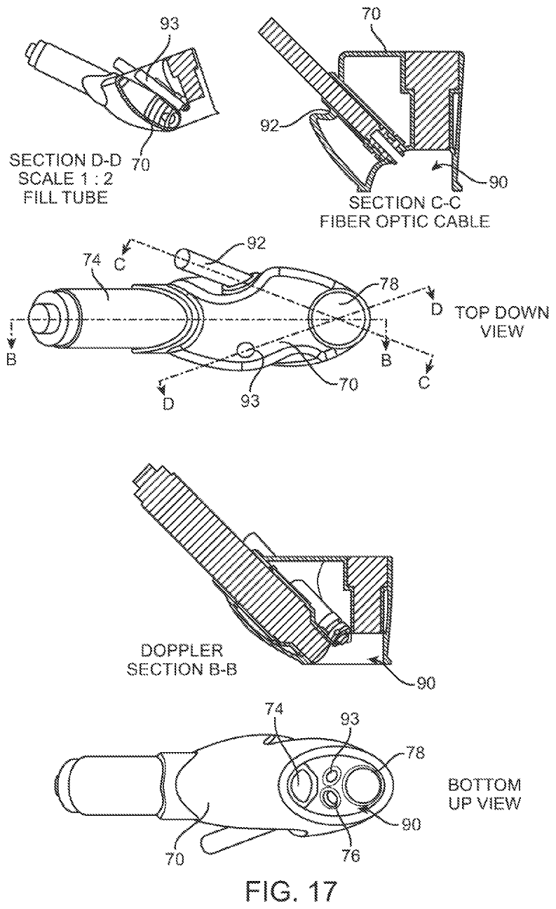

FIG. 17 provides engineering drawings of an embodiment including holder (70) which securely mounts ultrasound probe (74), fiber optic cable transmitting light (92) and optoacoustic probe (78). Also included in the depicted embodiments is a fill tube (93) for filling and refilling cavity (90) with acoustic gel.

Mode 3: Both ultrasound imaging and optoacoustic measurements are performed using the same ultrasound detector/array. In this shared mode, first ultrasound imaging is performed using the ultrasound array. Then, light pulses directed to the blood vessel of interest generate optoacoustic waves in the blood vessel and these optoacoustic waves are detected by the ultrasound array. Co-utilization of an ultrasound imaging probe as a detector of optoacoustic waves provides both ultrasound guidance for monitoring and detect optoacoustic waves induced in tissues (including blood vessels) by the optical sources. First, the blood vessel of interest is found using the standard ultrasound imaging mode which is based on generating ultrasound in the probe, directing it to the tissue, and detecting ultrasound echo signals from the tissues. Once the blood vessel is found, optical radiation is directed to the tissue. The optoacoustic waves generated in tissues propagate to the ultrasound probe and the ultrasound-sensitive detectors of the ultrasound probe detect the optoacoustic waves from the tissue. Then the optoacoustic signals are recorded and analyzed by the ultrasound system to display oxygenation.

Although each of these modes has advantages and drawbacks, they all may be used for ultrasound-guided optoacoustic monitoring depending on a specific application, position of the blood vessel, and its geometry. Whether used in any one the modes, in certain embodiments, ultrasound in a frequency range of 1-18 MHz is utilized for location of the vessel to be tested for oxygen saturation by optoacoustics. In other embodiments, the ultrasound in a frequency range of 4-13 MHz is utilized for location of the vessel to be tested for oxygen saturation by optoacoustics. In certain embodiments, ultrasound at a frequency 13±1 MHz is utilized for location of the vessel to be tested for oxygen saturation by optoacoustics.

Note that the term “Doppler” is used herein interchangeably with ultrasound (U/S) as Doppler utilizes ultrasound. “Doppler” has become synonymous with “velocity measurement” in medical imaging but as used herein, Doppler is used interchangeably with ultrasound. Where the term “Doppler” is used herein, it is because the U/S probe is specifically adapted to have velocity measurement capabilities although this is not required. Ultrasound imaging systems typically also have Doppler capabilities, so they provide both ultrasound imaging and velocity measurements in blood vessels in the images. The Doppler system in FIG. 15 is not an imaging system, it provides audible signals without images when its probe is directed to a blood vessel. So, using this audible signal, one may direct optoacoustic probe in optimal direction and at optimal location in the body. Imaging may be employed if desired.

Many optical sources with wavelengths suitable for oxygenation measurements may be used in the optoacoustic systems, including but not limited to: optical parametric oscillators (OPOs), laser diodes, light emitting diodes (LEDs), dye lasers, and solid-state lasers (such as Nd:YAG laser, Alexandrite laser). In certain embodiments, the light source may comprise one or more laser diodes or light emitting diodes. The light source of the monitor may be configured to generate light having an energy of 1 μJ to 1 mJ. The light source of the monitor may be configured to generate light having wavelengths in range of two or more of 685-715 nm, 715-745 nm, 745-775 nm, 790-820 nm, or 845-875 nm, such as two or more of 700 nm, 730 nm, 760 nm, 800 nm, 805 nm, or 860 nm, for example. Light from the source is conveyed such as via cables that comprise one or more optical fibers configured to direct light generated by the light source to the light output of the probe head.

Many acoustic detectors may be used in the optoacoustic systems, including but not limited to: piezodetectors that are based on piezomaterials such as piezopolymers and piezoceramics, capacitive micromachined ultrasonic transducers (CMUTs), and optically-based ultrasound detectors such as interferometric detectors, optical beam deflecting detectors, pressure-sensitive optical elements. In certain embodiments, the acoustic detector may further comprise an amplifier for the acoustic transducer. The probe head may further comprise an electromagnetic shield that shields the acoustic sensor and amplifier from electromagnetic interference. The probe head may further comprise an acoustic attenuator configured to absorb undesired ringing in the probe head.

In some embodiments, the acoustic detector comprises a piezoelectric transducer that uses the piezoelectric effect to measure changes in pressure, acceleration, strain, or force and convert them into an electrical signal. The sensor may be separated from the electromagnetic shield by a spacer element, which may be made of a polymeric material, such as polyamide. In some embodiments, the spacer element is approximately 0.005 to 5 mm thick.

The electrical signals generated by the acoustic sensor are transmitted to a Printed Circuit Board (“PCB”) via one or more electrical wires. The PCB includes a preamplifier that amplifies the signals received from the sensor before transmitting them to a monitor or computer of the system along further electrical wires. The preamplifier may be configured to provide about 40 dB of gain at about 500 kHz, having a bandwidth of about 3 dB in the range from about 40 kHz to about 10 MHz. The PCB may further comprise a digitizer configured to digitize the acoustic signal detected by the acoustic sensor. For example, the digitizer may be configured to sample the acoustic signal from the preamplifier at least at about 20 MHz, in response to a trigger signal from the laser diode subsystem connected to the probe, as described herein. The digitizer may, for example, store about 1000 samples of the acoustic signal, and transfer the block of samples to the processor of the console unit connected to and controlling the operation of the optoacoustic probe, for waveform averaging of the samples. An acoustic backing material may be positioned behind the acoustic sensor. It provides backing for the sensor (for wideband detection of pressure waves) and absorbs the vibrations that travel through the sensor to prevent undesired ringing in the signal and separate part of the signal from ringing noise. In some embodiments, the attenuator comprises a mass of epoxy. A hollow interior space, within which the internal components of the probe reside, may be substantially cylindrical, with a diameter in a range from about 8 to about 10 mm, and a height of about 10 mm.

The probe may be designed to reduce areas that are hard to clean and disinfect between uses, such as grooves or pockets of in the exterior surface of the housing. Alternatively, or in combination, the probe may comprise a disposable cover configured to be placed over the housing, in order to reduce the need for cleaning and disinfecting the probe between uses. The probe is preferably configured such that its components may withstand soaking in a disinfecting solution for sterilization.

In FIG. 4C the optoacoustic probe uses the piezoceramic lead zirconate titanate (Pb[Zr(x)Ti(1−x)]03) (“PZT”), 2 mm thick with a 3×3 mm area. In FIGS. 9A and 9B, the optoacoustic probe uses the piezopolymer polyvinylidene fluoride (“PVDF”), 110 μm thick with a 4×6 mm area. A specially designed miniature preamplifier is built in the probe with a bandwidth (at −3 dB level) 40 kHz<f<10 MHz. In FIG. 12, the optoacoustic probe uses PVDF, 110 μm thick, 6 mm diameter, with the preamplifier. In FIG. 13D, the optoacoustic probe uses PVDF, 52 μm thick, 2×3 mm area, with the preamplifier. In FIGS. 14A and 14B, the optoacoustic probe uses PVDF, 110 μm thick, 7 mm diameter. In FIGS. 15A-15C, the optoacoustic probe uses PVDF, 110 μm thick, 8 mm diameter. The probe is incorporated into the small oval holder for combination with the Doppler U/S probe (Huntleigh).

In certain embodiments, the optoacoustic system includes a console unit and a handheld probe. In certain embodiments the console unit includes a controller, a processor, a photodiode array, an acoustic processing subsystem, and a cooling subsystem. The probe directs optical signals from a light source such as an optical parametric oscillator (OPO), laser diode, light emitting diode (LED), pulsed laser diode, dye laser, or solid-state lasers (such as a Nd:YAG laser, Alexandrite laser) to subject tissue. The probe further comprises an acoustic transducer that receives acoustic signals generated in response to the directed light signals.

The processor may be configured to determine oxygenation of the subject in response to the measured acoustic pressure. The programmer may be programmed to provide one or more steps of the detection method, and the program may comprise program instructions stored on a computer readable memory or programmed steps of the logic circuitry such as programmable array logic or a field programmable gate array, for example. For measurement of oxygen saturation, formulas are applied to measure oxygenation when signals are good (i.e., there is low background). Theoretically, any wavelengths at which oxyhemoglobin and deoxyhemoglobin have different absorption may be used for oxygenation measurements. To measure oxygenation, at least two wavelengths are used. In certain embodiments a three-wavelength approach is utilized (760, 800, and 850 nm).

In certain embodiments, wavelengths of 760 nm and 800 nm are used. This pair is good because there is a big difference in oxy- and deoxyhemoglobin absorption spectra at 760 nm, while 800 nm is the reference point because oxy-and deoxyhemoglobin have the same absorption (isosbestic point).

In certain embodiments, the pair of wavelengths is 1064 nm and 800 nm because of a big difference of oxy- and deoxyhemoglobin absorption at 1064 nm. In still other embodiments, 760 nm and 1064 nm are utilized because at both wavelengths there is a big difference in oxy- and deoxyhemoglobin absorption.

In general, for any wavelength, i:

Rᵢ=aᵢ+bᵢ×SO₂

To derive the three-wavelength algorithm, subtracting 1.0 from both sides generates a difference of signals:

$${R}_{i} - 1 = {a}_{i} + {b}_{i} \times {SO}_{2} - 1$$

$$\frac{{A}_{i}}{{A}_{800}} - 1 = \frac{{A}_{i} - {A}_{800}}{{A}_{800}} = {a}_{i} + {b}_{i} \times {SO}_{2} - 1$$

We refer to the difference Aᵢ−A₈₀₀ as the differential amplitude, and label it Dᵢ. To determine the differential amplitude at 760 nm, D₇₆₀, we plug in the absorption coefficients at 760 nm from the absorption spectrum of blood to get the equation:

$$\frac{{D}_{760}}{{A}_{800}} = {a}_{760} + {b}_{760} \times {SO}_{2} - 1 = 2.02 - 1.31 \times {SO}_{2} - 1 = 1.02 - 1.31 \times {SO}_{2}$$

Similarly, at 1064 nm, we get:

$$\frac{{D}_{1064}}{{A}_{800}} = {a}_{1064} + {b}_{1064} \times {SO}_{2} - 1 = - 0.23 + 1.45 \times {SO}_{2} - 1 = - 1.23 + 1.45 \times {SO}_{2}$$

To remove A₈₀₀, Eq. 1 may be divided by Eq. 2 as follows.

$$\frac{{D}_{760}}{{D}_{1064}} = \frac{1.02 - 1.31 \times {SO}_{2}}{- 1.23 + 1.45 \times {SO}_{2}}$$

Solving for SO₂ yields:

$${D}_{760} ( - 1.23 + 1.45 \times {SO}_{2} ) = {D}_{1064} ( 1.02 - 1.31 \times {SO}_{2} )$$

$${SO}_{2} ( 1.45 {D}_{760} + 1.31 {D}_{1064} ) = 1.23 {D}_{760} + 1.02 {D}_{1064}$$

$${SO}_{2} = \frac{1.23 {D}_{760} + 1.02 {D}_{1064}}{1.45 {D}_{760} + 1.31 {D}_{1064}}$$

Since the absorption of melanin at 1064 nm is low, this last equation for SO2 can be used to measure oxygenation in subjects with high background from hair or skin melanin.

The last above equation for SO₂ may be used to measure oxygenation using any (bad or good) signals with high background from hair or skin melanin. Therefore, in certain embodiments, one, two, three or more wavelengths of light signals or two or more wavelength pairs for light signals may be used to measure oxygenation optoacoustically, even in conditions of high background. The wavelengths noted above are examples only, and other wavelengths are also contemplated for use as described above and herein. The above coefficients for the various formulas and equations are examples only as well, and other coefficients for the above formulas and equations are also contemplated for use.

The console may further comprise a power supply coupled to the optical subsystem, the acoustic sensor subsystem, and the processor. The console may further comprise a display coupled to the processor to display the determined oxygenation to a user. The display may comprise a touch screen for operating the console. The console may further comprise a housing enclosing the laser diode subsystem, the acoustic sensor subsystem, and the processor. The console may further comprise a second cooling fan, which may be coupled to one or more of the processor or acoustic sensor subsystem, for cooling the console. The processor may be capable of accessing medical records of the subject.

The console may further comprise an output port for the optical source such as the laser diode subsystem and an input port for the acoustic sensor subsystem. The output port and the input port may be configured to be coupled to a sensor module or an optoacoustic probe to emit the one or more light pulses to the tissue of the subject and to receive the acoustic pressure generated in the tissue. The output port and the input port may be configured to be coupled to the sensor module or optoacoustic probe with a cable comprising one or more optical fibers. The cooling subsystem may include a temperature controller that may include a temperature sensor to measure the temperature of the light source and a first thermoelectric cooler to add or remove heat to regulate the temperature of the light source in response to the measured temperature. While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure.

What is claimed is:

1. An apparatus for measurement of blood oxygenation of a subject or a blood vessel of the subject, the apparatus comprising:
(a) a base configured for placement against a portion of a body of a subject; and
(b) an adapter rotatably coupled to the base, the adapter comprising a housing configured to removably couple to one or more of an ultrasound probe or an optoacoustic probe such that said one or more of the ultrasound probe or optoacoustic probe is rotatable at a plurality of angles relative to the portion of the body.

2. The apparatus of claim 1, further comprising an anchor coupled to the base, wherein the adapter is rotatably coupled to the anchor.

3. The apparatus of claim 1 or 2, further comprising a shaft extending from the adapter and at least partially through a cavity within the anchor, the shaft configured to rotate within the cavity.

4. The apparatus of any one of claims 1 to 3, wherein the shaft rotates in concert with the adapter.

5. The apparatus of any one of claims 1 to 4, wherein the shaft is detachably coupled with the adapter.

6. The apparatus of any one of claims 1 to 5, wherein the anchor is detachably coupled with the base.

7. The apparatus of any one of claims 1 to 6, further comprising a locking mechanism configured to lock a rotational position of the adapter relative to the base.

8. The apparatus of any one of claims 1 to 7, wherein the locked rotational position of the adapter corresponds to a desired angle of the plurality of angles between the ultrasound probe, an optoacoustic probe, or both and the portion of the body.

9. The apparatus of any one of claims 1 to 8, wherein the locking mechanism comprises a clamp that clamps the shaft with the anchor, such that the adapter is prevented from further rotation.

10. The apparatus of any one of claims 1 to 9, wherein the ultrasound probe is coupled with an ultrasound probe extension.

11. The apparatus of any one of claims 1 to 10, wherein the optoacoustic probe is coupled with an optoacoustic probe extension.

12. The apparatus of any one of claims 1 to 11, wherein the adapter further comprises a proximal opening at the proximal end.

13. The apparatus of any one of claims 1 to 12, wherein the proximal opening is configured to receive the ultrasound probe, the ultrasound probe extension, the optoacoustic probe, the optoacoustic probe extension, or a combination thereof.

14. The apparatus of any one of claims 1 to 13, wherein at least a distal portion of the ultrasound probe, the ultrasound probe extension, the optoacoustic probe, the optoacoustic probe extension, or a combination thereof comprise the same form factor.

15. The apparatus of any one of claims 1 to 14, wherein the base comprises an adhesive to attach to at least one portion of the body of the subject.

16. The apparatus of claim 15, wherein the adhesive is a two-sided adhesive.

17. The apparatus of any one of claims 1 to 16, further comprising a second base coupled to a second anchor, the second base and second anchor coupled to the adapter via a second shaft.

18. The apparatus of any one of claims 1 to 17, further comprising a first screw configured to clamp the shaft to the anchor, and a second screw configured to clamp the second shaft to the second anchor, thereby preventing further rotation by the adapter.

19. The apparatus of any one of claims 1 to 18, wherein the portion of the body comprises a chest region and/or a neck region.

20. The apparatus of claim 19, wherein the portion of the body comprises an upper chest region and/or a lower neck region.

21. The apparatus of any one of claims 1 to 20, wherein, the housing is configured to removably couple to the ultrasound probe and subsequently removably couple to the optoacoustic probe after the ultrasound probe has been removed from the housing.

22. The apparatus of any one of claims 1 to 21, wherein one or more of the ultrasound probe or optoacoustic probe is rotatable toward a target blood vessel of the subject.

23. A system for measurement of blood oxygenation of a subject or a blood vessel of the subject, the system comprising:
(a) an ultrasound probe;
(b) an optoacoustic probe;
(c) a system controller operatively coupled to the ultrasound probe, the optoacoustic probe, or both;
(d) an apparatus configured to align one or both of the ultrasound probe and optoacoustic probe with at least one portion of the body of the subject, the apparatus comprising:
(i) a base configured for placement against the at least one portion of the body of the subject;
(ii) an adapter rotatably coupled to the base, the adapter comprising a housing configured to removably couple to one or more of the ultrasound probe or the optoacoustic probe, such that said one or more of the ultrasound probe or the optoacoustic probe is rotatable at a plurality of angles relative to the at least one portion of the body of the subject.

24. The system of claim 23, further comprising an anchor coupled to the base, wherein the adapter is rotatably coupled to the anchor.

25. The system of any one of claims 23 to 24, further comprising a shaft extending from the adapter and at least partially through a cavity within the anchor, the shaft configured to rotate within the cavity.

26. The system of claim 25, wherein the shaft rotates in concert with the adapter.

27. The system of claim 25, wherein the shaft is detachably coupled with the adapter.

28. The system of any one of claims 23 to 27, wherein the anchor is detachably coupled with the base.

29. The system of any one of claims 23 to 28, further comprising a locking mechanism configured to lock a rotational position of the adapter relative to the base.

30. The system of any one of claims 21 to 29, wherein the locked rotational position of the adapter corresponds to a desired angle of the plurality of angles between the ultrasound probe, an optoacoustic probe, or both and the at least one portion of the body of the subject.

31. The system of claim 29, wherein the locking mechanism comprises a clamp that clamps the shaft with the anchor, such that the adapter is prevented from further rotation.

32. The system of any one of claims 23 to 31, wherein the ultrasound probe is coupled with an ultrasound probe extension.

33. The system of any one of claims 23 to 32, wherein the optoacoustic probe is coupled with an optoacoustic probe extension.

34. The system of any one of claims 23 to 33, wherein the adapter further comprises a proximal opening at the proximal end.

35. The system of any one of claims 23 to 34, wherein the proximal opening is configured to receive the ultrasound probe, the ultrasound probe extension, the optoacoustic probe, the optoacoustic probe extension, or a combination thereof.

36. The system of any one of claims 23 to 35, wherein at least a distal portion of the ultrasound probe, the ultrasound probe extension, the optoacoustic probe, the optoacoustic probe extension, or a combination thereof comprise the same form factor.

37. The system of any one of claims 23 to 36, wherein the base comprises an adhesive to attach to the at least one portion of the body of the subject.

38. The system of claim 37, wherein the adhesive is a two-sided adhesive.

39. The system of any one of claims 23 to 38, further comprising a second base coupled to a second anchor, the second base and second anchor coupled to the adapter via a second shaft.

40. The system of any one of claims 23 to 39, further comprising a first screw configured to clamp the shaft to the anchor, and a second screw configured to clamp the second shaft to the second anchor, thereby preventing further rotation by the adapter.

41. The system of any one of claims 23 to 40, further comprising a display interface operatively coupled to the system controller, the display interface configured to display an image captured by the ultrasound probe and/or blood oxygenation measurement data via the optoacoustic probe.

42. The system of any one of claims 23 to 41, wherein the system controller is configured to activate and/or deactivate the ultrasound probe and/or the optoacoustic probe.

43. The system of any one of claims 23 to 42, wherein the system controller is operatively coupled to a subject/user interface.

44. The system of any one of claims 23 to 43, wherein the at least one portion of the body comprises a chest region and/or a neck region.

45. The system of claim 44, wherein the at least one portion of the body comprises the chest region and wherein the chest region comprises an upper chest region, or wherein the at least one portion of the body comprises the neck region and wherein the neck region comprises a lower neck region.

46. A method for measurement of blood oxygenation of a subject or a blood vessel of the subject, the method comprising:
(a) placing a base against a first portion of a body of a subject, wherein an adapter is rotatably coupled to the base;
(b) aligning an ultrasound probe with the first portion of the body or a second portion of the body via the adapter;
(c) adjusting an angle of alignment between the ultrasound probe and the first portion or the second portion of the body via rotation of the adapter to a first rotational position relative to the base, so as to identify a location of a blood vessel;
(d) based on an image obtained from the ultrasound probe, identifying the location of the blood vessel;
(e) aligning an optoacoustic probe at said angle of alignment with the first portion or the second portion, via the adapter that remains at the first rotational position; and
(f) measuring the blood oxygenation within the blood vessel with the optoacoustic probe.

47. The method of claim 46, wherein prior to step (d), based on an image obtained from the ultrasound probe that does not identify the location of the blood vessel, further adjusting the angle of alignment between the ultrasound probe and the first portion or the second portion of the body via rotation of the adapter to a second rotational position relative to the base.

48. The method of claim 46, wherein prior to step (d), based on an image obtained from the ultrasound probe that does not show an optimum angle of alignment for optoacoustic measurement of blood oxygenation in the blood vessel, further adjusting the angle of alignment between the ultrasound probe and the first portion or the second portion of the body via rotation of the adapter to a second rotational position relative to the base.

49. The method of any one of claims 46 to 48, wherein prior to step (d), based on identifying no rotational positions of the adapter available to identify the blood vessel, moving the base to another location on the first or second portion of the body.

50. The method of any one of claims 46 to 49, wherein prior to step (d), based on identifying no rotational positions of the adapter available to obtain an optimum angle of alignment for optoacoustic measurement of blood oxygenation in the blood vessel, moving the base to another location on the first portion or second portion of the body.

51. The method of any one of claims 46 to 50, wherein prior to aligning the optoacoustic probe, the rotational position of the adapter is locked via a locking mechanism, so as to prevent further rotation of the adapter relative to the base.

52. The method of claim 51, wherein the locking mechanism comprises a clamp that clamps the shaft with the anchor, such that the adapter is prevented from further rotation.

53. The method of any one of claims 46 to 52, wherein prior to aligning the optoacoustic probe, the base is secured to the first portion or the second portion of the body.

54. The method of any one of claims 46 to 53, wherein placing the base against the at least one portion of the body comprises adhering the base to the first portion of the body.

55. The method of any one of claims 46 to 54, wherein aligning the ultrasound probe comprises at least partially inserting the ultrasound probe within the adapter.

56. The method of any one of claims 46 to 54, wherein aligning the ultrasound probe comprises at least partially inserting the ultrasound probe into an ultrasound probe extension (UPE), and at least partially inserting the UPE within the adapter.

57. The method of claim 56, wherein aligning the optoacoustic probe comprises removing the ultrasound probe and/or UPE from the adapter, and at least partially inserting the optoacoustic probe within the adapter.

58. The method of claim 56, wherein aligning the optoacoustic probe comprises removing the ultrasound probe and/or UPE from the adapter, and at least partially inserting the optoacoustic probe into an optoacoustic probe extension (OPE), and at least partially inserting the OPE within the adapter.

59. The method of any one of claims 46 to 58, wherein the image is obtained via the ultrasound probe by using a system controller to activate said ultrasound probe.

60. The method of any one of claims 46 to 59, wherein the image is displayed on a display interface operatively coupled to the system controller.

61. The method of any one of claims 46 to 60, wherein measuring the blood oxygenation comprises using a system controller to activate said optoacoustic probe.

62. The method of claim 61, further comprising displaying the blood oxygenation of the blood vessel using a display interface operatively coupled to the system controller.

63. The method of any one of claims 46 to 62, wherein one or more of the first portion of the body and the second portion of the body comprises a chest region and/or a neck region.

64. The method of claim 63, wherein one or more of the first portion of the body and the second portion of the body comprises an upper chest region and/or a lower neck region.

65. The method of any one of claims 46 to 64, wherein the blood vessel comprises a left innominate vein, a right innominate vein, a superior vena cava, an aorta, a right internal jugular vein, a left internal jugular vein, a left subclavian vein, a right subclavian vein, or a combination thereof.
